(19) United States
(12) Patent Application Publication
(10) Pub. No.: US 2015/0160121 A1
(43) Pub. Date: Jun. 11, 2015

(54) Calibration Transfer and Maintenance in Spectroscopic Measurements of Ethanol

(71) Applicants: Trent Daniel Ridder, Clovis, NM (US); Benjamin Ver Steeg, Redlands, CA (US)

(72) Inventors: Trent Daniel Ridder, Clovis, NM (US); Benjamin Ver Steeg, Redlands, CA (US)

(21) Appl. No.: 14/561,982

(22) Filed: Dec. 5, 2014

Related U.S. Application Data
(60) Provisional application No. 61/913,204, filed on Dec. 6, 2013.

(51) Int. Cl. G01N 21/25; A61B 5/00

(52) U.S. Cl. CPC G01N 21/25; A61B 5/4845

(57) ABSTRACT

Methods of producing a plurality of spectroscopic measurement devices, comprising producing a calibration model that includes the expected range of measurement variation across the plurality of devices; producing the devices; installing the calibration model on each device. Most standard methods focus on ways to reduce the number of replicate samples that are required to be taken on a given instrument or class of instruments. The present methods can reduce that number to zero by anticipating the expected range of instrument variation in manufacturing in the field. This can be important when measuring live biological samples as it is impractical to maintain standard humans, cells, etc. This is in contrast to measurements on dry agricultural products where a standard, sealed dry sample can be maintained for months/years when required.

### CROSS REFERENCE TO RELATED APPLICATIONS

This application claims priority to U.S. provisional application 61/913,204, filed Dec. 6, 2013, which is incorporated herein by reference.

### SUMMARY OF THE INVENTION

Example embodiments of the present invention provide methods of producing a plurality of spectroscopic measurement devices, comprising: (a) producing a calibration model that includes the expected range of measurement variation across the plurality of devices; (b) producing the devices; (c) installing the calibration model on each device.

Such methods can further comprise determining the expected range of measurement variation from an analytical model of the device.

In such methods, producing a calibration model can comprise: collecting one or more base calibration spectra on a base instrument; producing a plurality of synthetic calibration spectra from the base calibration spectra with a transfer function determined from the device design; and producing the calibration model from the base calibration spectra and the synthetic calibration spectra.

In such methods, the spectroscopic measurement device can be one or more of: a Fourier transform spectrometer, a dispersive spectrometer, a filter based spectrometer, a laser-based spectrometer, and an LED-based spectrometer.

In such methods, the expected range of measurement variation can include variation due to one or more of: wavelength axis, line shape, resolution, intensity shifts, noise frequency content, and noise frequency bandwidth.

In such methods, the expected range of measurement variation can include variation due to manufacturing tolerances in the optical interface with the sample.

Example embodiments of the present invention can provide a spectroscopic measurement device, having a calibration model produced according to the methods described.

### BRIEF DESCRIPTION OF THE DRAWINGS

FIG. 1 is a schematic diagram of the Michelson interferometer used in the present work.

FIG. 2 is an illustration of the effects of self apodization at several wavenumbers for an interferometer with a constant 3.6 degree divergence half angle for the collimated beam operating at 32 cm-1 resolution.

FIG. 3 is an illustration of weighting (window A) and phase (window B) functions versus optical path difference obtained from equations 8-11 at 1000 wavenumber intervals from 4000 to 8000 cm-1.

FIG. 4 is an illustration of an example of the effects of shear where a constant 1 micron misalignment between the two retroreflectors is present throughout the interferometer scan (e.g. the shear, s, is constant for all OPD).

FIG. 5 is an illustration an interferogram (grey line) obtained using a 1.5 μm (6,667 cm-1) HeNe laser and an interferometer of the design of the present work that was intentionally misaligned to induce a large shear.

FIG. 6 is an illustration two cases of laboratory data obtained from an interferometer of the design used in this work.

FIG. 7 is an illustration of instrument specific variable shear as a function of OPD.

FIG. 8 is an illustration of the effect of variation in shear on instrument line shape.

FIG. 9 is an illustration of the effects of shear and off axis detector FOV relative to the case where only self apodization due to a finite sized light source is present.

FIG. 10 is an illustration of PCA factors (Window A) and log screen plot (Window B) for spectra shown in Window A of FIG. 9.

FIG. 11 is pictorial representation of an objective of the modification process.

FIG. 12 is a diagram of an interferogram modification process.

FIG. 13 is an illustration of the background corrected normal spectra (Window A), background corrected MIMIK spectra (Window B), and their difference (Window C).

FIG. 14 is an illustration of the root mean squared error of cross validation (RMSECV) obtained from Partial Least Squares (PLS) regression for the four cases.

FIG. 15 is an illustration of ethanol prediction bias by instrument for each of the four cases.

FIG. 16 compares the Mahalanobis distance and spectral F-ratio metrics obtained for the validation data for the normal calibration data, no background correction case (Case A, solid black line) and the MIMIK calibration data with background correction case (Case D, solid grey line).

FIG. 17 is an illustration of the RMSECV curves obtained for three cases, all of which employed background correction: the normal data as both the calibration and validation set, the normal data predicting the MIMIK data, and the MIMIK data as both the calibration and validation set.

### DESCRIPTION OF THE INVENTION

The present description references several publications, patents, and other references. Each of those is incorporated herein by reference.

Part 1: Mathematical Basis for Spectral Distortions in FTNIR

Multivariate calibration transfer in spectroscopy is an active area of interest. Many current approaches rely on the measurement of a subset of calibration samples on each instrument produced. In many applications the measurement of subsets of calibration samples is not practical. Furthermore, such methods attempt to model implicitly, rather than explicitly, inter-instrument differences. In Part 1 of this description, an FTNIR system designed to perform noninvasive ethanol measurements is described. Optical distortions caused by self apodization, shear, and off axis detector field of view (FOV) are examined and equations describing their effects are given. The effects of shear and off axis detector FOV are shown to yield nonlinear distortions of the amplitude and wavenumber axis in measured spectra that cannot be accommodated by typical wavenumber calibration procedures or background correction. The distortions forecast by these equations are verified using laboratory measurements and an analysis of the spectral complexity caused by the distortions is presented. The theoretical and experimental aspects presented in Part I are incorporated into new calibration transfer methods whose benefits are illustrated using noninvasive alcohol measurements in Part 2 of this description.

The present description investigates multivariate calibration transfer for noninvasive spectroscopic ethanol measurements. The noninvasive alcohol measurement employs Fourier Transform near-infrared (FTNIR) spectroscopy in the 4000 to 8000 cm⁻¹ spectral region, which is of interest for noninvasive alcohol measurements because it offers specificity for a number of analytes, including alcohol and other organic molecules, while allowing optical path lengths of several millimeters through tissue, thus allowing penetration into the dermal tissue layer where alcohol is present in the interstitial fluid. G. L. Cote, “Innovative Non- or Minimally-Invasive Technologies for Monitoring Health and Nutritional Status in Mothers and Young Children,” Nutrition, 131, 1590S-1604S (2001). H. M. Heise, A. Bittner, and R. Marbach, “Near-infrared reflectance spectroscopy for non-invasive monitoring of metabolites,” Clinical Chemistry and Laboratory Medicine, 38, 137-45 (2000). V. V. Tuchin, Handbook of Optical Sensing of Glucose in Biological Fluids and Tissues, CRC press (2008). Several publications have discussed the underlying near infrared spectroscopic method (T. D. Ridder, S. P. Hendee, and C. D. Brown, “Noninvasive Alcohol Testing Using Diffuse Reflectance Near-Infrared Spectroscopy,” Applied Spectroscopy, 59(2), 181-189 (2005). T. D Ridder, C. D. Brown, and B. J. Ver Steeg, “Framework for Multivariate Selectivity Analysis, Part II: Experimental Applications,” Applied Spectroscopy, 59(6), 804-815 (2005)) and its clinical comparison to blood and breath alcohol assays. T. Ridder, B. Ver Steeg, and B. Laaksonen, “Comparison of spectroscopically measured tissue alcohol concentration to blood and breath alcohol measurements,” Journal of Biomedical Optics, 14(5), (2009). T. Ridder, B. Ver Steeg, S. Vanslyke, and J. Way, “Noninvasive NIR Monitoring of Interstitial Ethanol Concentration,” Optical Diagnostics and Sensing IX, Proc. of SPIE Vol. 7186, 71860E1-11 (2009). T. D. Ridder, E. L. Hull, B. J. Ver Steeg, B. D. Laaksonen, “Comparison of spectroscopically measured finger and forearm tissue ethanol to blood and breath ethanol measurements,” Journal of Biomedical Optics, pp. 028003-1-028003-12, 16(2), 2011. The present description investigates and evaluates an approach to calibration transfer that achieves acceptable performance while avoiding the use of methodologies that would be prohibitive due to the nature of noninvasive alcohol tests.

Calibration transfer, calibration standardization, and transfer of calibration all relate to the same problem: the process/method/techniques associated with making a calibration obtained from one (or one set) of spectrometers valid on a second (or second set) of spectrometers. Several review articles discuss the various calibration transfer approaches employed by researchers. O. E. DeNoord, “Multivariate Calibration Standardization,” Chemometrics and Intelligent Laboratory Systems, 25(2), p. 85-97, 1994. R. N. Feudale, N. A. Woody, H. W. Tan, A. J. Myles, S. D. Brown, J. Ferre, “Transfer of multivariate calibration models: a review,” Chemometrics and Intelligent Laboratory Systems, 64(2), p. 181-192, 2002. T. Fearn, “Standardization and calibration transfer for near infrared instruments: a review,” Journal of Near Infrared Spectroscopy, 9(4), p. 229-244, 2001. For example, deNoord discusses univariate and multivariate near infrared (NIR) calibrations, the general problem of calibration standardization, strategies and approaches for achieving effective calibration transfer, and spectral preprocessing approaches including derivatives, bias correction, and wavelength selection. Fearn discusses calibration standardization and transfer as well as three potential approaches: development of robust calibrations, adjusting spectra via transformations such as direct standardization or piecewise direct-standardization, and spectral preprocessing methods such as wavelength selection, derivatives, sample selection, and scatter correction. It is important to note that the utility of the various approaches to calibration transfer depends strongly on the specific application under consideration and that multiple approaches are often used in conjunction in many applications.

Several commonly employed calibration transfer methodologies require a subset of the calibration samples to be measured on each device in order to determine a spectral transform. The transform is applied to either the calibration data or to future validation data with the objective of making the calibration and validation data more similar to each other. The transform often takes the form of a series of coefficients or a matrix of coefficients where the transformed response at a given wavelength is a weighted combination of the original spectrum at several wavelengths. As such, the transformation approaches can be thought of as a convolution whose kernel can vary with wavelength, which allows them to accommodate sources of spectroscopic variation such as wavelength shifts and lineshape changes that simple approaches such as background correction cannot.

While transforms can be effective for some applications, they are limited in the sense that they offer minimal insight into the underlying optical phenomena that cause problematic spectral variation between instruments. At the most basic level, differences in optical components and their alignment alter the propagation of light through the spectrometer system. How those alterations manifest in measured spectra depends strongly on the spectrometer design. Understanding how instrument design, optical components and alignment tolerances impact measured spectra is important as it can provide insight that is desirable for two reasons. First, such insight can allow refinement of the instrument design, fabrication, and alignment process in order to improve uniformity and thereby directly reduce the problematic sources of spectral variance. Second, calibration transfer approaches can be implemented that explicitly address the problematic spectral distortions. In some cases, such knowledge can be used to a priori determine the types of spectroscopic variation a given practitioner can expect to encounter between multiple spectrometers. We propose that this type of information can be effectively used in the formation of the calibration and obviate the need to acquire a subset of calibration spectra on each device produced.

The present description is presented in two parts. The first part presents a summary of the FTNIR spectrometer system used in the noninvasive alcohol measurement system, which is then used to develop a mathematical basis for several types of spectral distortions that can be observed between instruments. The derived spectral distortions are then compared to spectroscopic measurements acquired in the laboratory in order to verify that the derived equations yield spectral distortions that can be directly observed in physical instrumentation. The second part provides a description of a spectral modification method based on the mathematical foundations from part 1 that is used to alter clinical calibration data. The impact of those modifications on multivariate calibration transfer in noninvasive ethanol measurements is described.

Ideal Interferometers and the Consequences of Finite Sized Light Sources

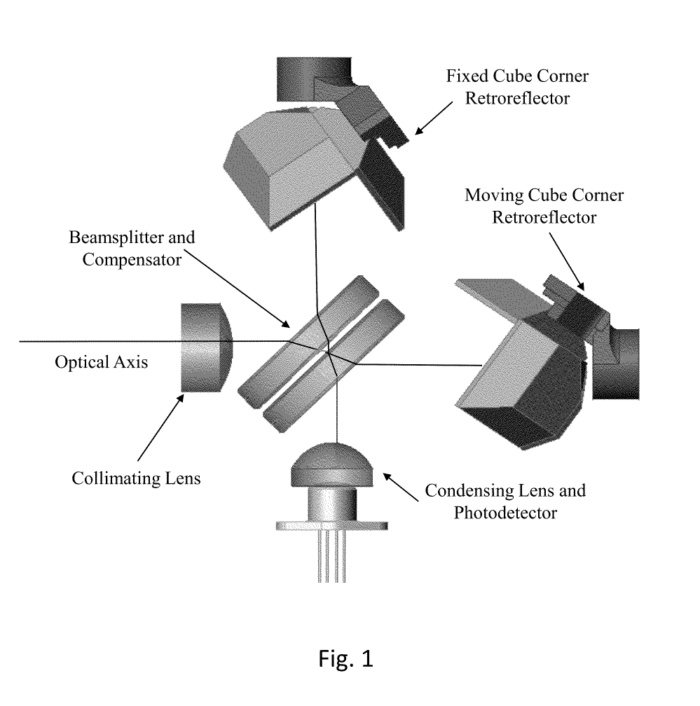

The spectrometer system used in this description uses a Michelson geometry interferometer operating in the NIR (4000-8000 cm⁻¹) at 32 cm⁻¹ resolution. The interferometer, schematically shown in FIG. 1, uses cube-corner retroreflectors due to their reduced sensitivity to misalignment relative to flat mirrors. The underlying theory of cube-corner retroreflectors and their advantages and disadvantages relative to flat mirrors can be found elsewhere. P. Griffiths, J. de Haseth, Fourier Transform Infrared Spectrometry, Wiley-Interscience, 1986. E. R. Peck, Theory of the Cube Corner Interferometer, Journal of the Optical Society of America, pp. 1015-1024, 38(12), 1948. E. R. Peck, Uncompensated Corner-Reflector Interferometer, Journal of the Optical Society of America, pp. 250-252, 47(3), 1957. Regardless of the choice of flat mirrors or retroreflectors, the purpose of the interferometer is to determine the spectrum associated with light introduced at its input (e.g. intensity versus wavenumber). An ideal interferometer accomplishes this by modulating different wavenumbers of light to different frequencies according to:

F(x)=∫_{-∞}^{∞}B(σ)e^{i2πσx}dσ, (1)

where F is the intensity measured at the detector, x is the optical path difference (OPD) and B(s) is the spectral intensity at wavenumber s. F(x) is called the interferogram, the Fourier transform of which yields the desired intensity versus wavenumber spectrum. Equation 1 is simplistic in the sense that it assumes an “ideal” interferometer. The line shape of an ideal Michelson interferometer is determined by the range of optical path differences, x, induced by the travel of the moving mirror. Longer distances of travel correspond to a more narrow line shape (e.g. higher resolution). However, ideal interferometers do not exist and as such equation 1 does not fully represent the measured signal in practical interferometers.

One requirement of ideal interferometers is that the beam of light passing through it must be perfectly collimated. In practice, only an infinitely small point source can be perfectly collimated. Unfortunately, an infinitely small light source is neither possible nor would it allow measurements with any reasonable signal to noise ratio (SNR). As such, all practical interferometers seek to collimate light collected from a source of finite size. This, in turn, implies that the light travelling through the interferometer is not perfectly collimated. A consequence of imperfectly collimated light passing through an interferometer is referred to as self-apodization, which has been previously described. S. P. Davis, M. C. Abrams, J. W. Brault, Fourier Transform Spectrometry, Academic Press, 2001. J. Chamberlain, The Principles of Interferometric Spectroscopy, Wiley, 1979. G. A. Vanasse and H. Sakai, “Fourier Spectroscopy, Chapter 7”, Progress in Optics, vol 6, pp. 261-332, North-Holland Publishing Company, Amsterdam, 1967. P. Griffiths, J. de Haseth, Fourier Transform Infrared Spectrometry, Wiley-Interscience, 1986. The two primary effects of self apodization are a weighting of the intensity of the interferogram (eq. 2) and an alteration to the wavelength axis of the spectrum (eq. 3). The intensity weighting is given by:

$$A ( x , σ ) = sin c ( \frac{x σ}{2 π} Ω ) , \qquad ( 2 )$$

where A(x,s) is the weighting caused by self apodization as a function of optical path difference (x) in cm and wavenumber (s), and Ω is the solid angle of the imperfectly collimated beam. The solid angle is given by Ω=πρ₀², where ρ₀ is the divergence half angle of the collimated beam in radians.

The effective optical path difference, xₑ, is given by:

$${x}_{e} = x ( 1 - \frac{Ω}{4 π} ) , \qquad ( 3 )$$

Note that equation 3 indicates that the effective optical path difference (xₑ) is linearly related to optical path difference (x), which results in linearly multiplicative shift in the location of features in the measured spectrum (e.g. the shift at 8000 cm⁻¹ is twice the shift at 4000 cm⁻¹). As a result, the change in the wavelength axis caused by self apodization is easily accommodated by a wavelength calibration procedure.

Equation 1 can be re-written to include the effects of self apodization:

$$F ( x ) = {\int }_{- ∞}^{∞} B ( σ ) sin c ( \frac{x σ}{2 π} Ω ) {e}^{i2πσ x} ( 1 - \frac{Ω}{4 π} ) d σ , \qquad ( 4 )$$

Note that in the case of perfect collimation (Ω=0) equation 4 simplifies to the ideal case shown in equation 1. Substituting equations 2 and 3 into equation 4 and rearranging yields:

F(x)=∫_{-∞}^{∞}B(σ)A(x,σ)e^{i(2πσx-φ(x,σ))}dσ, (5)

### Where

$$φ ( x , σ ) = σ x \frac{Ω}{2} , \qquad ( 6 )$$

Note that as equations 2-6 are written the solid angle, and therefore divergence angle, is independent of wavenumber. This may or may not be true in a given instrument depending on whether effects such as chromatic aberration are present. Regardless, equation 5 shows that the effects of self apodization on the interferogram are given by an amplitude weighting, A(x,σ), and a phase shift, φ(x,σ), both of which depend on wavenumber, optical path difference, and the angular divergence of light through the interferometer.

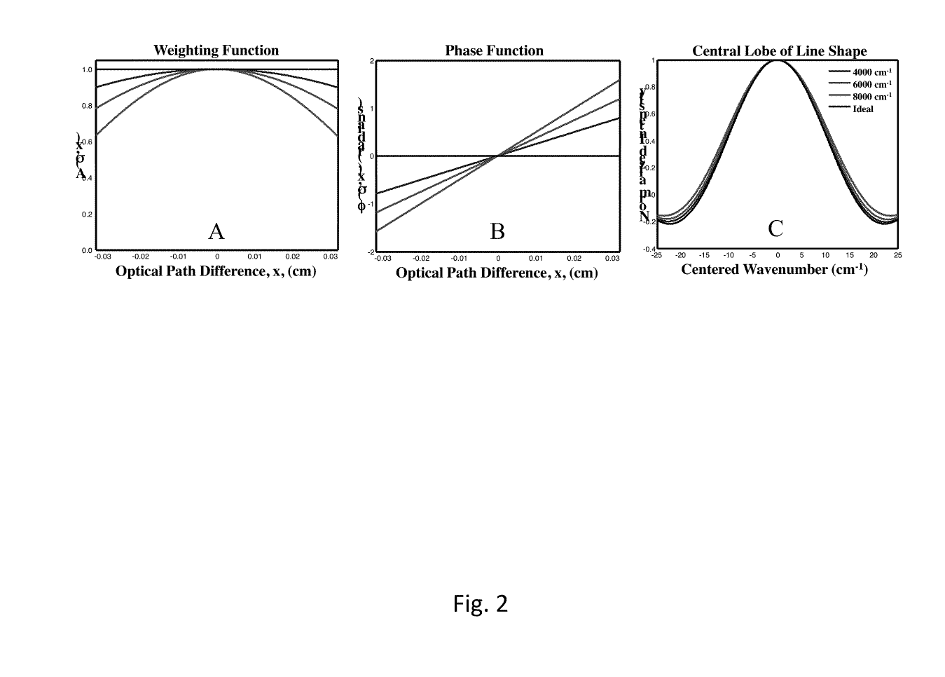

FIG. 2 shows the effects of self apodization at several wavenumbers for an interferometer with a constant 3.6 degree divergence half angle for the collimated beam operating at 32 cm⁻¹ resolution. These correspond to the nominal divergence and resolution of the interferometer design of this work. Window A of FIG. 2 illustrates the weighting functions versus optical path difference obtained from equation 2, highlighting that the effects of self apodization become more pronounced as wavenumber increases. The window B of FIG. 2 shows the phase functions obtained from equation 6. It is important to note that the slope of the linear component of the phase function is directly proportional to the wavenumber shift observed in the spectral domain. Thus, increased phase slope corresponds to a greater wavenumber shift and, similar to the weighting function, the effect increases with wavenumber.

Window C of FIG. 2 demonstrates the effect of the weighting function on the central lobe of the instrument line shape. To facilitate comparison, the central wavenumber of each line has been subtracted from each line and the peak height has been normalized. In other words, neither the absolute change in peak intensity nor the wavenumber shift of each line is shown. The line shape of each wavenumber is broadened relative to the ideal, no self apodization, case with higher wavenumbers exhibiting greater broadening. In aggregate, FIG. 2 highlights an important consideration of self-apodization: its effect on the instrument line shape is not constant with wavenumber, even in the case of a constant divergence half angle.

Note that self-apodization is expected and cannot be avoided as finite light sources must be used in any practical instrument. Therefore some beam divergence must be present and it is up to the practitioner to determine an appropriate balance of light source size, which increases throughput and signal to noise ratio (SNR), and beam divergence, which degrades resolution and can exacerbate optical alignment challenges. Furthermore, differences in divergence half angle between instruments, for example due to variations in the alignment of the collimating lens, will yield instrument specific variations in the wavenumber dependent instrument line shape, location, and intensity. As a result, from a calibration transfer perspective, an objective is to keep the spectral manifestations of self apodization as constant as possible unit to unit and then accommodate any residual variation in self apodization within the multivariate calibration.

Other Important Sources of Amplitude Weighting and Phase Shifts

Equation 5 is generally applicable to any alterations to the interferogram caused by changes in the angular distribution of light passing through the interferometer. As such, an intermediate result of the present description is to obtain a more comprehensive set of equations for A(x,σ) and φ(x,σ) that include other important sources of inter-instrument variations. In addition to self apodization, there are other optical parameters and effects in a Michelson interferometer that can alter the range of angles measured by the photodetector. As such they will have their own contribution to the weighting, A(x,σ), and phase functions, φ(x,σ), of the interferogram. However, there is no expectation that the contributions will be of the form shown in equations 2 and 3.

Two important considerations in a Michelson interferometer with cube corner retroreflectors are misalignment of the detector field of view (FOV) relative to the interferometer optical axis and shear (misalignment of one or both of the cube corner retroreflectors with respect to the optical axis or each other). Appendices A and C of Hearn provide a comprehensive treatment of these effects, respectively. D. R. Hearn, Fourier Transform Interferometry, Technical Report 1053, Lincoln Laboratory, Massachusetts Institute of Technology, Lexington, Mass., 1999. Additional supporting information on the mathematical solutions provided by Hearn can be found elsewhere. M. V. R. K. Murty, Some More Aspects of the Michelson Interferometer with Cube Corners, Journal of the Optical Society of America, pp. 7-10, 50(1), 1960. K. W. Bowman, H. M. Worden, R. Beer, Instrument line shape modeling and correction for off-axis detectors in Fourier transform spectrometry, Jet Propulsion Laboratory, 1999. H. M. Worden, K. W. Bowman, Tropospheric Emission Spectrometer (TES) Level 1B Algorithm Theoretical Basis Document, v. 1.1, JPL D-16479, Jet Propulsion Laboratory, 1999. M. Born, E. Wolf, Principles of Optics, 7th edition, Cambridge University Press, 1999. A brief summary of the equations relevant to this work is below.

Off Axis Detector Field of View

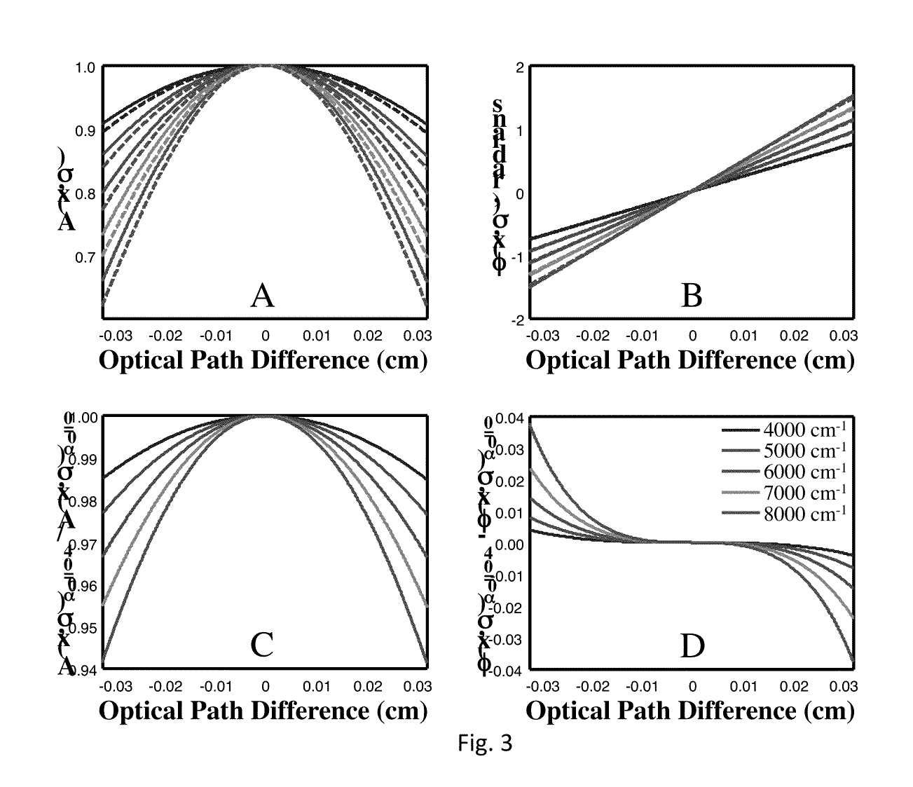

Substituting equation A-2 from Hearn into equation 14b from Hearn gives the following equation for an interferogram measured in the presence of an off-axis detector.^{Error! Bookmark not defined.}

$$F ( x , {α}_{0} , {ρ}_{0} ) = 2 {\int }_{0}^{∞} {B}_{σ} ( 1 + \frac{1}{Ω} {\int }_{- π}^{π} {\int }_{0}^{{ρ}_{0}} cos ( C cos ( ρ ) + S sin ( ρ ) cos ( β ) ) sin ( ρ ) d ρ d β ) d σ , \qquad ( 7 )$$

where C=2πσx cos(α₀), S=2πσx sin(α₀), ρ is the elevation of a given ray from the center of the detector FOV, and β is the azimuthal angle of a given ray from the center of the FOV. F(x,α₀,ρ₀) is the interferogram as a function of optical path difference when the detector FOV is displaced an angle, α₀, from the optical axis and the collimated beam has a divergence half angle, ρ₀. See FIG. 3 of Hearn for a graphical representation of the optical geometry encompassed by equation 7. Note that, unlike earlier equations in this work, equation 7 is in cosine, rather than complex, form. In any case, the presence of sine terms in equation 7 is indicative that phase effects are present when the detector FOV is off-axis.

For the purposes of this work, it is preferable to express the optical effects described by equation 7 in terms of weighting, A(x,σ), and phase, φ(x,σ) as discussed above. After considerable manipulation, Hearn (D. R. Hearn, Fourier Transform Interferometry, Technical Report 1053, Lincoln Laboratory, Massachusetts Institute of Technology, Lexington, Mass., 1999) and Murty (M. V. R. K. Murty, Some More Aspects of the Michelson Interferometer with Cube Corners, Journal of the Optical Society of America, pp. 7-10, 50(1), 1960) arrive at the following equations for the weighting function (The solution to the integrals within Hearn are in terms of Lommel functions. There are two solutions, referred to as Un and Vn, only one of which is valid in a given situation. In Hearn's application one solution was valid at all evaluated points for the FT system under consideration. As a result, the second solution was not included. However, Murty shows both Lommel solutions as well as the means to determine which is valid for a given value of u and w (p and q in Murty). Murty also provides the reduced solution in the case that either u or w (p or q) is zero):

$$\begin{matrix}A ( x , σ ) = \frac{2}{u} \sqrt{{U}_{1}^{2} - {U}_{2}^{2}} for \vert \frac{u}{w} \vert \leq 1 ; & ( 8 ) \\ A ( x , σ ) = \frac{2}{u} \sqrt{\begin{matrix}1 + {V}_{0}^{2} + {V}_{1}^{2} - 2 {V}_{0} cos ( \frac{u}{2} + \frac{{w}^{2}}{2 u} ) - \\ 2 {V}_{1} sin ( \frac{u}{2} + \frac{{w}^{2}}{2 u} )\end{matrix}} for \vert \frac{u}{w} \vert > 1 , & ( 9 )\end{matrix}$$

and the phase function:

$$\begin{matrix}φ ( x , σ ) = \frac{u}{2} - {tan}^{- 1} ( \frac{{U}_{2}}{{U}_{1}} ) for \vert \frac{u}{w} \vert \leq 1 ; & ( 10 ) \\ φ ( x , σ ) = \frac{u}{2} + {tan}^{- 1} ( \frac{{V}_{0} + cos ( \frac{u}{2} + \frac{{w}^{2}}{2 u} )}{{V}_{1} - sin ( \frac{u}{2} + \frac{{w}^{2}}{2 u} )} ) for \vert \frac{u}{w} \vert > 1 , & ( 11 )\end{matrix}$$

where u=2πσx cos(α₀)sin²(ρ₀) and w=2πσx sin(α₀)sin(ρ₀). Uₙ and Vₙ are the Lommel functions defined as:

$$\begin{matrix}{U}_{n} = {\sum }_{i = 0}^{∞} {( - 1 )}^{i} {( \frac{u}{w} )}^{2 i + n} {J}_{2 i + n} ( w ) \\ and & ( 12 ) \\ {V}_{n} = {\sum }_{i = 0}^{∞} {( - 1 )}^{i} {( \frac{w}{u} )}^{2 i + n} {J}_{2 i + n} ( w ) & ( 13 )\end{matrix}$$

where n is the order of the Lommel function, i is the current term of the series expansion being computed, and J₂ᵢ₊ₙ is the Bessel function of order 2i+n. In general, we have found that three terms (max i of 2 in the integrals of equations 12 and 13) is sufficient to calculate the weighting and phase functions with sufficient accuracy. Interpretation of the weighting and phase functions from equations 8-11 is not straightforward and is best shown graphically using information from the interferometer design used in the present work.

FIG. 3 shows weighting (window A) and phase (window B) functions versus optical path difference obtained from equations 8-11 at 1000 wavenumber intervals from 4000 to 8000 cm⁻¹. In all cases, the divergence half angle, ρ₀, was 3.6 degrees. The solid lines in windows A and B represent the case where the detector FOV is on-axis (α₀=0). In this case, equations 8-11 reduce to equations 2 and 6. The dashed lines in windows A and B represent the case where the detector FOV is offset from the interferometer's optical axis by 0.4 degrees, which, for the interferometer design of this work corresponds to a translation of the detector FOV relative to the optical axis of approximately 1 mm.

The dashed lines represent the case where self apodization and the distortion caused by detector misalignment are simultaneously present while the solid lines include only the effects of self apodization. At first glance, the differences between the solid and dashed lines might seem subtle. Windows C and D show the isolated effects of the off-axis detector FOV that were obtained by dividing the dashed lines by the solid lines for A(x,σ) and subtracting the solid lines from the dashed lines for φ(x,σ) (If multiple sources of amplitude weighting are present, they can be combined by multiplying the associated A(x,σ)'s. Likewise, individual sources can be isolated from a combined A(x,σ) via division. Phase terms are additive rather than multiplicative and are therefore isolated via subtraction of φ(x,σ)'s). The concept of relative comparisons is used in several places throughout this work in order to isolate the distortions caused by specific instrument non-idealities from unavoidable phenomena such as self apodization. Importantly, the residual phase in window D of FIG. 3 is a nonlinear function of both optical path difference and wavenumber. This corresponds to a distortion of wavenumber axis of the instrument line shape. The distortion is comprised of a shift in the location of the line caused by the linear component of the phase function and higher order distortions caused by the nonlinear portion of the phase function. From a calibration transfer perspective, inter-instrument variation in the location of the detector FOV will yield spectral differences that cannot be compensated by a simple wavelength calibration procedure or background correction.

Alignment of Cube Corner Retroreflectors

Considerable literature exists that compares the merits of interferometers incorporating flat mirrors versus those incorporating cube corner retroreflectors. P. Griffiths, J. de Haseth, Fourier Transform Infrared Spectrometry, Wiley-Interscience, 1986. E. R. Peck, “Theory of the Corner-Cube Interferometer,” Journal of the Optical Society of America, pp. 1015-1024, 38(12), 1948. E. R. Peck, Uncompensated Corner-Reflector Interferometer, Journal of the Optical Society of America, pp. 250-252, 47(3), 1957. One of the primary advantages of cube corner retroreflectors is that, unlike flat mirrors, they are insensitive to tilts in alignment. However, they are instead sensitive to the alignment of each retroreflector vertex to the interferometer optical axes. One impact of misalignment of retro reflector vertices is referred to as shear and an extensive discussion of the types of shear can be found elsewhere. W. H. Steele, Interferometry, Chapter 5, Cambridge University Press, New York, 1967.

For the purposes of this description, similar to the effects of off-axis detector FOV, the objective is to express the effects of shear in terms of weighting, A(x,σ), and phase, φ(x,σ), functions. The weighting and phase functions given by equations 8-11 are also applicable to a cube-corner misalignment of s cm, albeit with a re-definition of u and w (Equations 8-11 yield A(x,σ)=1 and φ(x,σ)=0 for all x, s, and α0 when Ω=0 (e.g. ρ0=0). In other words, an ideal interferometer, with its perfectly collimated beam, would not exhibit any effects from shear or an off-axis detector FOV. As such, they are rarely discussed in introductory interferometry texts.) (D. R. Hearn, Fourier Transform Interferometry, Technical Report 1053, Lincoln Laboratory, Massachusetts Institute of Technology, Lexington, Mass., 1999.):

u=2πσx sin²(ρ₀), (15)

and

w=4πσs sin(ρ₀), (16)

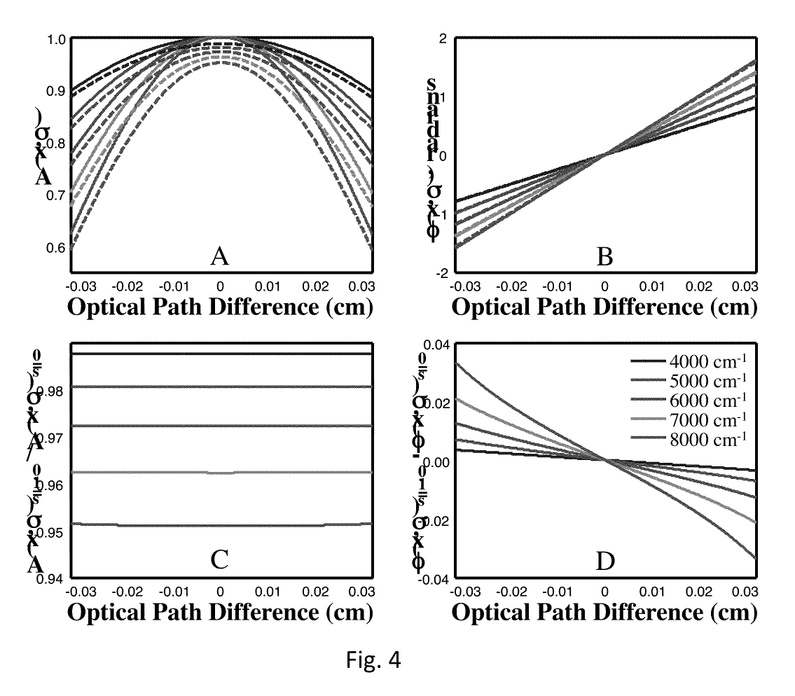

FIG. 4 shows an example of the effects of shear where a constant 1 micron misalignment between the two retroreflectors is present throughout the interferometer scan (e.g. the shear, s, is constant for all OPD). As with the detector FOV example, the divergence half angle is 3.6 degrees and the resolution is 32 cm⁻¹. The solid lines in the top windows of FIG. 4 show the weighting functions due to self apodization alone and the dashed lines include self apodization as well as the effects of the 1 micron shear. Windows C and D of FIG. 4 show the influence of shear alone.

The primary effect of shear on A(x,σ) is a suppression of intensity at all OPD's (the horizontal/constant part of each line) that becomes more pronounced as wavenumber increases. However, there is also a more subtle curvature present in each line that indicates a change in line shape accompanies the change in intensity. As with the overall intensity, the magnitude of the curvature increases with wavenumber. Furthermore, the bottom right window of FIG. 4 shows that a constant shear generates both a linear and nonlinear phase component in addition to the linear phase shift caused by self apodization. Although the manifestations of shear are somewhat different than those of detector FOV, the message is the same: variations in shear between instruments will correspondingly yield wavenumber dependent differences in intensity, line shape, and wavenumber axis distortion between instruments.

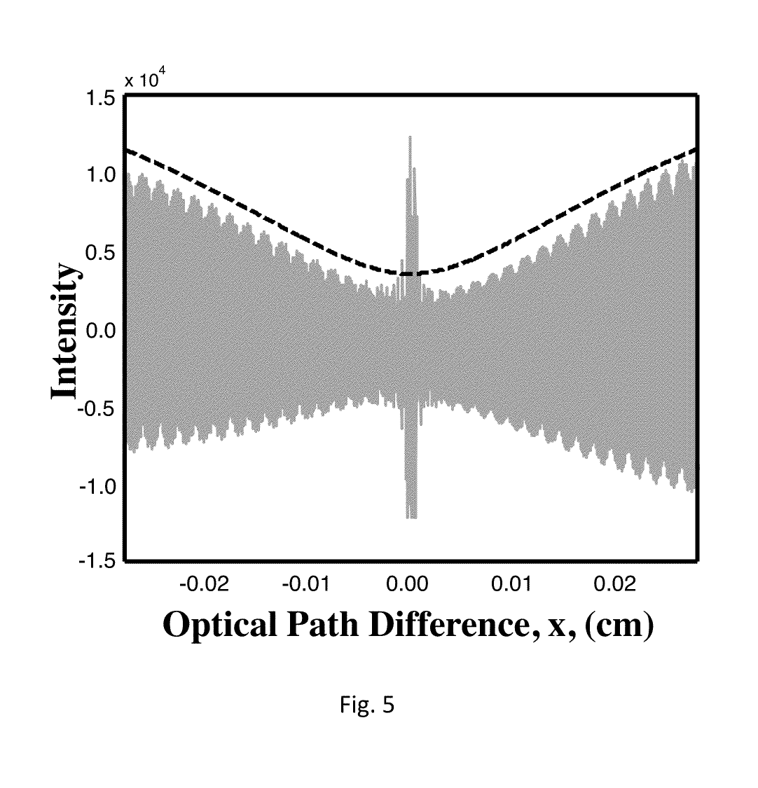

An interesting effect of shear is that, unlike self apodization and off-axis detector FOV, it has a stronger impact on the weighting function near zero path difference (ZPD). As a result, when shear is severe, the interferogram of a monochromatic light source can exhibit a “bowtie” effect. FIG. 5 shows an interferogram (grey line) obtained using a 1.5 μm (6,667 cm⁻¹) HeNe laser and an interferometer of the design of the present work that was intentionally misaligned to induce a large shear. Note that the HeNe interferogram also exhibits a small white light interferogram (the signal near x=0) which is caused by the blackbody self-emission of the optical components of the interferometer. The black, dashed line in FIG. 5 is the weighting function generated from equations 8 and 9 for 6,667 cm⁻¹ light using a constant shear (s) of 8 μm for all OPD, a divergence half angle of 3.6 degrees, and a resolution of 32 cm⁻¹. In addition to demonstrating the “bowtie” effect, FIG. 5 is also useful in the sense that it provides reassurance that the equations in this work generate weight and phase functions that can be replicated in laboratory measurements. FIG. 5 also suggests that the use of a monochromatic light source during interferometer alignment can provide useful information that a white light cannot. The interferogram of a white (or any broadband) light source varies strongly near ZPD but quickly loses intensity as absolute OPD increases. As such, a broad band light source does not allow the bowtie phenomenon to be observed.

An important, and more complex, aspect of shear from retro reflector alignment is that it is unlikely to be constant at all OPD's of an interferogram as the drive mechanism of the moving cube corner is unlikely to maintain constant retroreflector alignment throughout the scan. In other words, s can vary as a function of x in equation 16. Given the nature of mechanical mirror drives, the variation in s is unlikely to be random as a function of x but rather a slowly varying function that is based on the design of the drive mechanism. Likewise, there is no expectation of consistency in the variation of s between instruments. Regardless of the functional form of the variation in s, the impacts of shear caused by cube corner misalignment are not constant across the interferogram when such variation is present. This gives rise to several interesting phenomena in the resulting interferograms and spectra.

First, for light of a single wavenumber, the point of maximum intensity in the interferogram does not necessarily coincide with zero path difference. The result is the strange condition where the interferogram obtained from polychromatic light can have a maximum near the expected location of zero path difference yet have the maxima of the interferograms of individual wavenumbers of light displaced significantly from ZPD. Indeed, when such a situation is observed this is an indicator that variation in shear exists across the measured optical path differences. Note that this effect is not to be confused with chirping due to dispersion effects such as a mismatch of the beam splitter and compensator thicknesses as their origins and manifestations are different than those of shear.

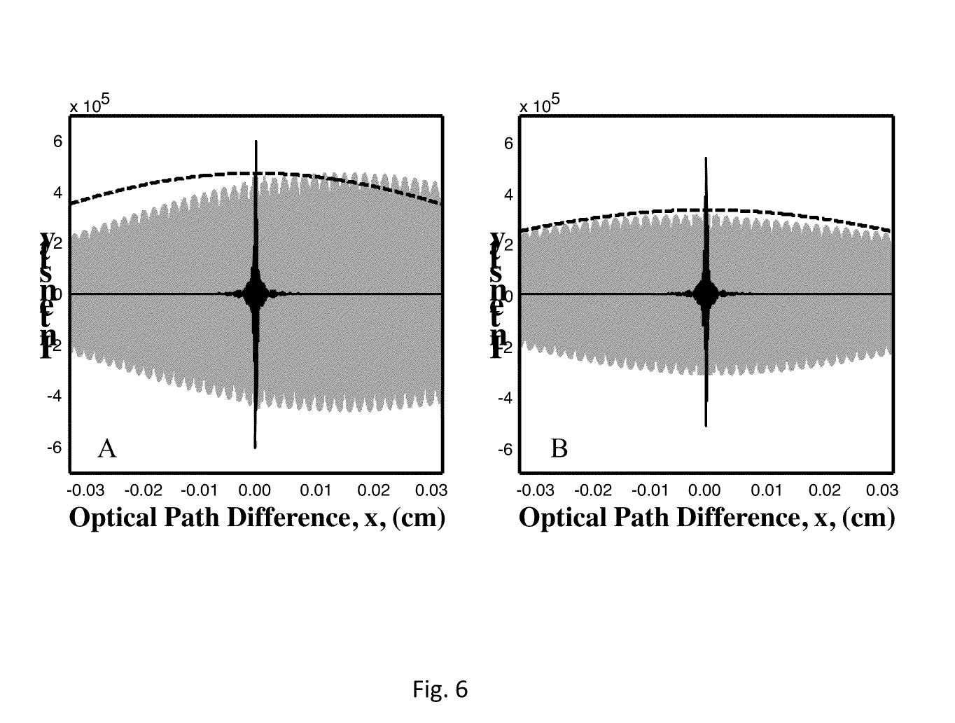

FIG. 6 shows two cases of laboratory data obtained from an interferometer of the design used in this work. Each case shows a white light interferogram as well as an interferogram obtained from a 1.5 micron (6,667 cm⁻¹) HeNe laser. Window A of FIG. 6 shows an interferometer with substantial shear variation across the range of measured OPD's (x). This is indicated by the displacement of the maximum of the HeNe interferogram relative to the maximum of the white light interferogram. Window B shows the same interferometer following realignment to better align the two maxima. For reference, the dashed line in both windows represents the weighting function, A(x,6667 cm⁻¹), solely due to the self apodization of a 3.6 degree diverging collimated beam which was calculated using equation 2. The weighting function has been scaled to the maximum intensity value of the 6,667 cm⁻¹ HeNe interferogram in each window. From a physical perspective, the window B corresponds to an interferometer alignment state where the moving retroreflector maintains more consistent alignment relative to the optical axis throughout its range of travel. Furthermore, the envelope of the 6,667 cm⁻¹ HeNe interferogram in window B is similar to that of the weighting caused by self apodization which is reassuring that the alignment is approaching a state where expected phenomenon such as self apodization are dominant. However, the alignment state shown in window B of FIG. 6 is undoubtedly imperfect as some variation in retroreflector alignment during the scan must invariably remain. As such, it is worth examining the spectral phenomena resulting from a variation in shear within a scan in more detail.

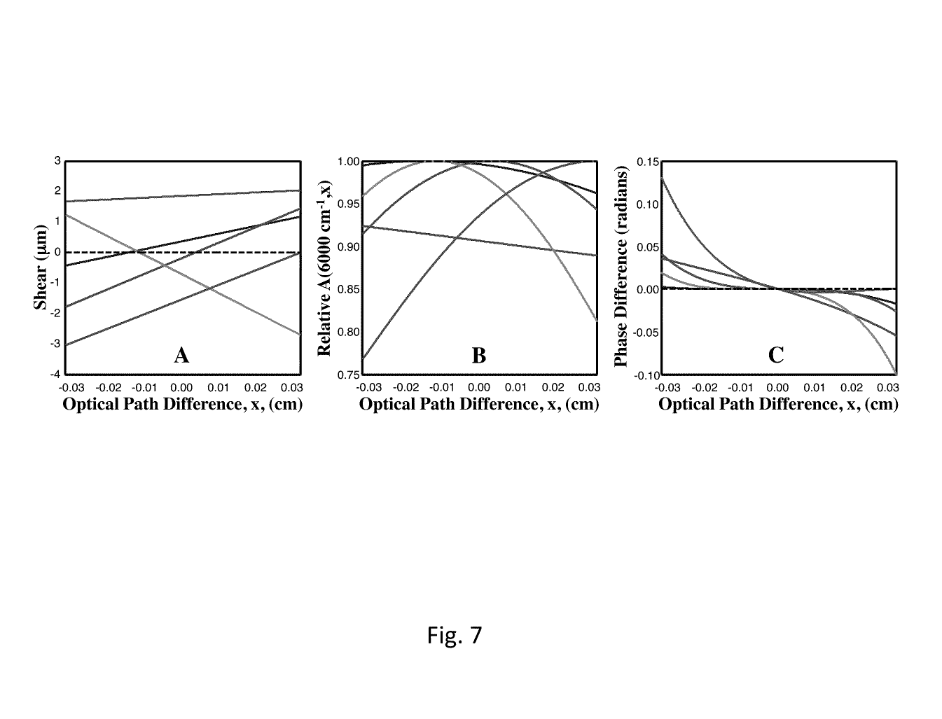

Window A of FIG. 7 shows shear as a function of optical path difference for several hypothetical interferometers (solid colored lines) as well as an ideally aligned interferometer (black dashed line). As with prior examples, the divergence half angle is 3.6 degrees and the resolution is 32 cm⁻¹. In this somewhat simplistic example, each hypothetical interferometer maintains a perfectly linear trajectory throughout the scan. However, each trajectory has an offset and angle from the optical axis of the interferometer. The result is a shear that linearly varies with optical path difference for each instrument. Window B shows the relative weighting functions for 6000 cm⁻¹ light, A(x, 6000 cm⁻¹), after normalizing by the ideally aligned case in order to remove the effects of self apodization. Unlike prior examples, the effects of a varying shear during a scan result in an asymmetric weighting function about ZPD. Similarly, window C of FIG. 7 shows the phase functions associated with the variable shear after subtracting the phase function of the ideally aligned case. The phase functions are both nonlinear and asymmetric which indicates the resulting wavenumber axis distortions in the spectral domain will be substantially more complicated than the multiplicative shift caused by self apodization alone. As a result, in contrast to the prior examples that broadened and altered the instrument line shape in a symmetric fashion, shear that varies with OPD will asymmetrically distort the instrument line shape in both the intensity and wavenumber domains. It is important to note that the physical distances involved in this example (single digit microns) are certainly in the realm of plausible based on practical mechanical and alignment tolerances. Furthermore, while this example assumes a perfectly linear trajectory for the moving mirror, quadratic and higher order variations in the trajectory could be present based on the type of mechanical drive used.

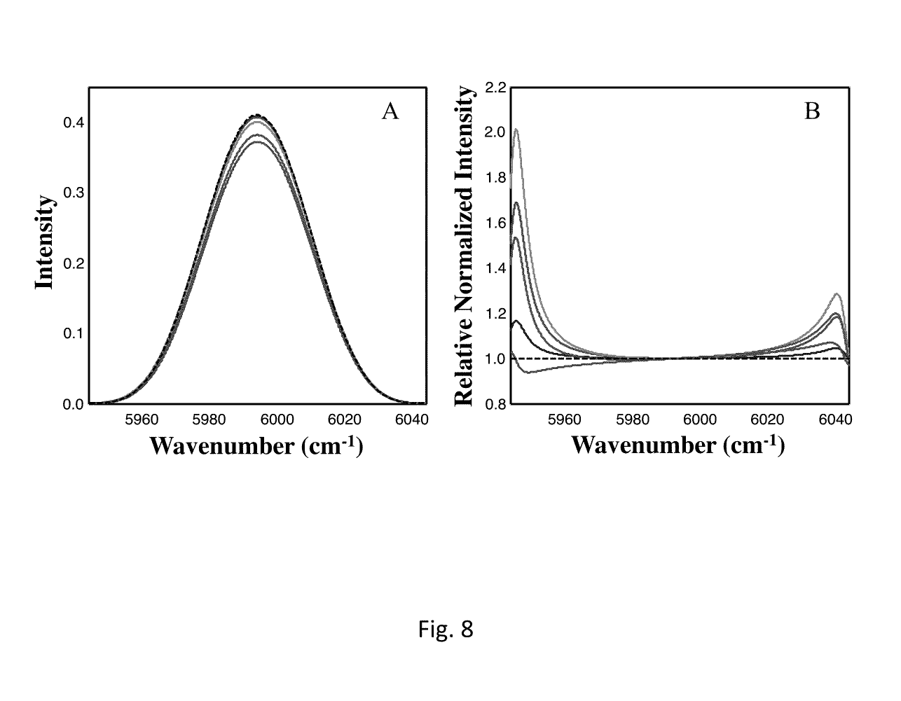

The example in FIG. 7 indicates that asymmetric distortions will occur to the instrument line shape when variation in shear occurs during an interferometer scan. In order to get a sense for how those distortions manifest in spectral space, interferograms of monochromatic 6000 cm⁻¹ light were generated for the weighting and phase cases shown in FIG. 7 using equation 6. The resulting interferograms were Blackman apodized in order to ease interpretation by suppressing the side lobes of the Sinc line shape prior to taking the Fourier transform. The resulting instrument line shapes are shown in window A of FIG. 8. The black dashed line represents the ideal perfectly aligned case where the only non-ideality present is self apodization caused by the imperfectly collimated beam. The solid, colored lines show the line shapes that result from the shear cases in FIG. 7. The dominant effect is suppression of intensity relative to the case where only self apodization is present. The asymmetric line shape distortions are more subtle and are shown in the window B of FIG. 8. Each line in the right window of FIG. 8 was obtained by normalizing the intensity of the line shape in the left window of FIG. 8 and dividing by the normalized ideal line shape. Clearly, the observed effects are not well described by a simple peak broadening/distortion and wavenumber shift.

Perspective: FTIR vs. FTNIR

NIR interferometers generally operate at more moderate resolutions (32 cm⁻¹ in this work) relative to their infrared (IR) counterparts as IR spectroscopy generally requires higher resolution due to the presence of sharper and more defined spectral features. The reduced resolution of NIR measurements translates to a shorter range of optical path differences in the interferogram that, in turn, allow a larger solid angle to pass through the interferometer before self apodization strongly impacts the resolution and instrument line shape. Thus, a larger source can be used and greater instrument throughput can be achieved relative to interferometers operating at higher resolution.

The prior sections demonstrate that this throughput advantage can come with several consequences as the distortions related to the alignment of the detector FOV and retroreflectors increase in magnitude as angular divergence through the interferometer and wavenumber increase. Thus, despite operating at lower resolution, tolerances on the alignment of optical components in FTNIR can be more stringent due to the larger wavenumbers in the NIR and the potentially increased solid angle of the collimated beam. For example, a mid-infrared (MIR) interferometer operating at 1 cm⁻¹ resolution with a maximum wavenumber of interest of 4000 cm⁻¹ and a divergence half angle of 0.016 radians (0.91 degrees) might have a cube corner alignment tolerance of 10 microns. The corresponding tolerance for a NIR interferometer operating at 32 cm⁻¹ resolution with a maximum wavenumber of interest of 8000 cm⁻¹ and a divergence half angle of 0.063 radians (3.6 degrees) would be 1.3 microns. A lesson from this example is that the balance between throughput and alignment tolerances depends on the resolution required by the application of interest as well as the wavenumber region employed.

Examination of the Spectral Distortions Caused by the Derived Weighting and Phase Functions

The prior examples presented the effects of self apodization, shear, and off-axis detector FOV at discrete wavenumbers; it has been shown that these effects are all wavenumber dependent. As a result, the true underlying A(x,σ) and φ(x,σ) for any measured interferogram are continuous surfaces. In addition to the examinations of the instrument line shape in prior examples it is also important to examine the distortions caused by shear, off axis detector FOV, and self apodization for a spectrum relevant to noninvasive ethanol measurements.

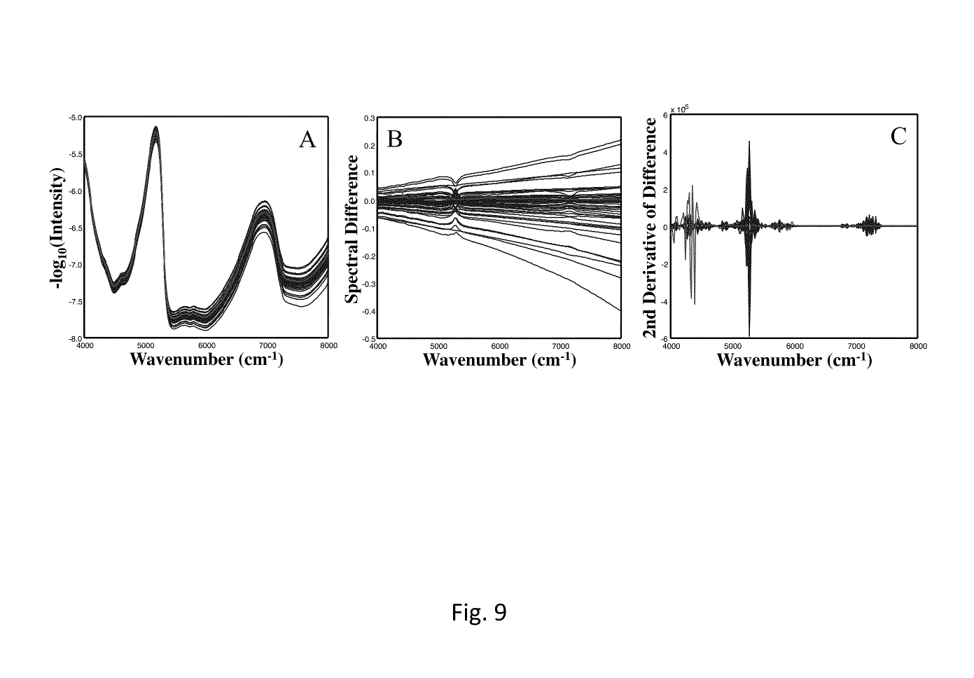

FIG. 9 is an example of the effects of shear and off axis detector FOV relative to the case where only self apodization due to a finite sized light source is present. The red trace in window A is the spectrum obtained from an interferogram determined using equation 4 and a divergence half angle of 3.6 degrees. B(σ) for this example was obtained by averaging noninvasive measurements acquired from the fingers of 106 people measured on 10 measurement devices of the same design. While the in vivo spectra undoubtedly contain unknown amounts of spectral distortions from the instruments on which the spectra were acquired, the purpose of the in vivo data in this example is solely to provide a relevant B(σ) for subsequent comparisons.

The blue traces in window A of FIG. 9 were obtained using the same B(σ). Each blue line corresponded to randomly chosen shear, detector alignment, and divergence half angle conditions within the intervals shown in table 1. Equations 8-11 and the randomly chosen conditions were used to calculate φ(x,σ) and A(x,σ) which were then used in conjunction with equation 5 to obtain interferograms (The description of the calculation of φ(x,σ), A(x,σ), and subsequent interferograms is admittedly somewhat sparse; Part II of this work provides the step by step process for determining and implementing φ(x,σ) and A(x,σ) surfaces to form interferograms). 200 interferograms and corresponding spectra were generated, 50 of which are shown in FIG. 9.

TABLE 1
Shear, off axis detector FOV, and angular divergence intervals

| Parameter | Description | Minimum | Maximum | Purpose |
|---|---|---|---|---|
| ρ₀ | Angular divergence | 3.4° | 3.8° | Simulates poor collimation |
| | of the collimated beam | | | lens alignment |
| | at 4000 cm⁻¹ | | | |
| S₁ | Shear at minimum | −4 mm | +4 mm | Retroreflector |
| | OPD (x) | | | misalignment, off axis |
| | | | | retroreflector trajectory |
| S₂ | Shear at maximum | −4 mm | +4 mm | Retroreflector |
| | OPD (x) | | | misalignment, off axis |
| | | | | retroreflector trajectory |
| α₀ | Angle of detector | 0° | 1° | Misalignment of the |
| | FOV displacement | | | detector FOV |
| | from the optical axis | | | |

Window B of FIG. 9 shows the spectral residuals obtained by subtracting the red trace from window A from each of the blue traces. As the underlying spectrum, B(σ), was constant in all cases, the residuals in window B of FIG. 9 are indicative of spectral distortions. There are two important considerations when examining the residuals. First, the parameter ranges in table 1 represent plausible variations in alignment that might be observed between instruments with reasonable optical and mechanical tolerances. Second, the magnitude of the residuals is large enough that it can be of significant concern depending on the spectroscopic application of interest.

In the present description, the NIR system is designed to perform noninvasive alcohol measurements. As such, the red trace in window B of FIG. 9 is a 10× magnification of a pure component spectrum of 80 mg/dL of ethanol (80 mg/dL is the legal driving limit in the United States) and a path length of 2 mm. Given that the ethanol signal is magnified by a factor of ten relative to the residuals it is straightforward to conclude that the spectral distortions caused by shear and detector alignment are certainly worth additional investigation with respect to their impact on noninvasive alcohol measurements.

The spectral residuals shown in window B of FIG. 9 are dominated by slightly quadratic component that is primarily caused by the constant component of shear (e.g. the portion of s that is independent of OPD, x). However, more subtle, higher frequency variation is also present. Window C of FIG. 9 shows the 2^{nd} derivative of the residuals in window B as well as the 2^{nd} derivative of the 10× magnified ethanol pure component spectrum. Together, windows B and C indicate the spectral distortions are both significant in magnitude as well as their complexity.

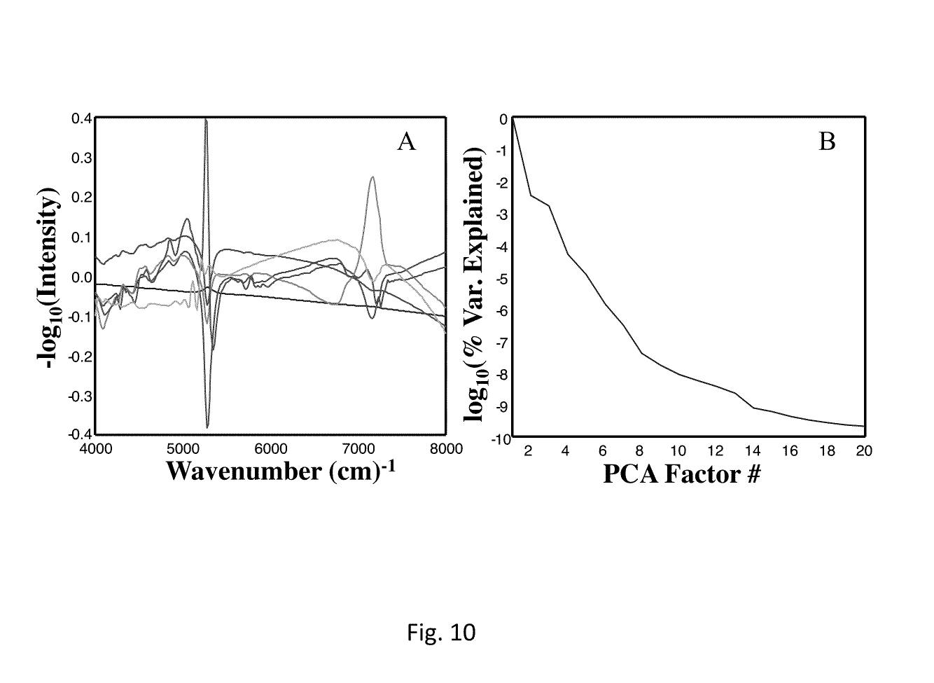

A Principal Components Analysis (PCA) was performed on the 200 generated spectra. As the same “true” spectrum, B(σ), was the input for the 200 spectra, the resulting principal components are comprised solely of the distortions caused by variation in self apodization, shear, and off axis detector FOV. Factors 1-6 of the PCA are shown in window A of FIG. 10 and the log screen plot for factors 1-20 is shown in window B of FIG. 10. Examination of window A shows that the first factor is a predominantly linear baseline that corresponds to the first order effect of shear. However, factors 2-6 exhibit significant spectral structure. The screen plot suggests that several of the factors explain appreciable variance, particularly as no noise is present in the decomposition. Certainly, the observed factors depend on the original B(σ) as the instrument distortions are based on the light input to the interferometer rather than introducing their own spectral signatures. As a result, the distortions will manifest differently for each sample measured and will therefore have a detrimental impact that must be mitigated by the employed multivariate calibration and calibration transfer techniques.

No attempt is being made to suggest that the pure component spectrum of ethanol in any way represents the in vivo ethanol signal measured in reflectance. However, the comparison is useful in the sense that the magnitude of spectral residuals is certainly large enough that the distortions caused by self apodization, shear, and detector FOV alignment are worthy of additional attention. Part II of this work focuses on a method for incorporating controlled amounts of the spectral distortions into clinical calibration data and evaluating the impact of their inclusion on multivariate calibration transfer in noninvasive ethanol measurements.

The present description has shown the origins of several important distortions to interferograms and spectra as well as laboratory methods for detecting their presence. As a result, the laboratory measurements and the equations presented in this work can be useful for diagnosing problematic instruments at the time of their alignment and remedying the associated cause prior to deployment. Furthermore, the present description supports methods for improving the interferometer alignment process beyond the use of a broad band light source that can also aid in the reduction in distortions observed between instruments. In other words, a significant benefit of the detection and correction of instruments exhibiting distortions caused by shear, off axis detector FOV, and undesirable variation in self apodization is that it condenses the range of spectral variation that calibration transfer methods must accommodate. Another benefit of the present work, is that not only can distortions be identified and corrected by realignment of the interferometer or replacing the offending optical component, but linkages to physical causes can provide useful design feedback that can reduce the range of spectral distortions in future revisions of the spectroscopic device.

The equations presented in this work can be expanded to include other sources of variation in angular distribution. For example, the weighting and phase surfaces for shear and off axis detector FOV are calculated independently in this work (both Phase I and Phase II) and then combined. However, it is possible that an interaction between the two could exist that this independent treatment ignores. As a result, the development of a single set of expressions that combines shear and off axis detector FOV can provide the ability to account for the potential interaction.

Another expansion area is an additional level of realism in the functional forms of the inputs to equations 8-11. For example, in the present description the variation in shear, s, as a function of OPD, x, was assumed to be linear. Solid models of the servo and flexure for the moving cube corner are readily generated by 3D modeling software. These models can provide a more accurate representation of the actual motion of the cube corner during the scan. As a result, any potential nonlinear motion such as a twist or rotation of the cube corner during its travel can be included in the determination of the shear distortion.

Interactions between the light source and the instrument are also an area of interest because the light source can impart angular and spatial structure to the light input to the interferometer. The present equations assume that the collimated beam, while diverging due to the light source's finite size, is spatially uniform and radially symmetric. That assumption can be violated in many circumstances and add an additional layer of complexity to the distortions caused by shear, self apodization, and off axis detector FOV. If could be possible to extend the framework presented in this work to accommodate a heterogeneous collimated beam by evaluating equations 8-11 for each location and angle in the collimated beam and combining them with appropriate weights.

Finally, as noted in the introduction, the present work involves calibration transfer of noninvasive ethanol measurements. A purpose of Part 1 was to establish a series of formulas that appropriately reflect real-world spectroscopic distortions when using an FTNIR spectrometer with cube corner retroreflectors. Part II leverages those formulas by using them as part of a calibration transfer methodology that modifies experimentally acquired data using the Part I formulas in a manner that represents the types of spectral variation that would be encountered over a broad population of instruments. Noninvasive alcohol measurements acquired from a controlled dosing study are used to demonstrate the advantages of the new methodology on multivariate calibration.

Part II: Modification of Instrument Measurements by Incorporation of Expert Knowledge (MIMIK)

Several calibration transfer methods require measurement of a subset of the calibration samples on each future instrument which is impractical in some applications. Another consideration is that these methods model inter-instrument spectral differences implicitly, rather than explicitly. The present description benefits from the invention that explicit knowledge of the origins of inter-instrument spectral distortions can benefit calibration transfer during the alignment of instrumentation, the formation of the multivariate regression, and its subsequent transfer to future instruments. In Part 1 of this description, a FTNIR system designed to perform noninvasive ethanol measurements was discussed and equations describing the optical distortions caused by self apodization, retroreflector misalignment, and off axis detector field of view (FOV) were provided and examined using laboratory measurements. The spectral distortions were shown to be nonlinear in the amplitude and wavenumber domains and thus cannot be compensated by simple wavenumber calibration procedures or background correction. Part 2 presents a calibration transfer method that combines in vivo data with controlled amounts of optical distortions in order to develop a multivariate regression model that is robust to instrument variation. Evaluation of the method using clinical data showed improved measurement accuracy, outlier detection, and generalization to future instruments relative to simple background correction.

Multivariate calibration methods such as Partial Least Squares (PLS), Principal Component Regression (PCR), and Multiple Linear Regression (MLR) are powerful techniques that enable quantitative analysis of analytes in complex systems using a variety of spectroscopies. However, their implementation requires a significant departure from univariate calibration approaches. In univariate calibrations, spectrometers are calibrated at a single wavelength of interest using a small set of calibration standards. Given this relatively small burden, each spectrometer can be independently calibrated at regular intervals. However, multivariate methods typically require a significantly larger quantity of calibration data because multiple variables are incorporated in the calibration model. This can make multivariate calibrations a time-consuming and resource intensive process which makes independent calibration of each device costly. Consequently, there is a strong desire to generate a multivariate calibration that is valid for all existing and future spectrometers.

Calibration transfer, calibration standardization, and transfer of calibration all relate to the same problem: the process, method, and techniques associated with making a calibration obtained from one or more of spectrometers valid on subsequent spectrometers. There are several review articles that discuss various calibration transfer approaches employed by researchers. O. E. DeNoord, “Multivariate Calibration Standardization,” Chemometrics and Intelligent Laboratory Systems, 25(2), p. 85-97, 1994. R. N. Feudale, N. A. Woody, H. W. Tan, A. J. Myles, S. D. Brown, J. Ferre, “Transfer of multivariate calibration models: a review,” Chemometrics and Intelligent Laboratory Systems, 64(2), p. 181-192, 2002. T. Fearn, “Standardization and calibration transfer for near infrared instruments: a review,” Journal of Near Infrared Spectroscopy, 9(4), p. 229-244, 2001. For example, deNoord discusses univariate and multivariate calibrations and multiple strategies and approaches for achieving effective calibration transfer. Furthermore, Fearn discusses three general approaches to calibration standardization and transfer; the formation of robust calibrations, spectral transformations such as direct standardization or piecewise direct-standardization, and spectral preprocessing methods such as wavelength selection, derivatives, background correction, and scatter correction. Both deNoord and Fearn note that the utility of the various approaches to calibration transfer depends strongly on the specific application under consideration and that multiple approaches are often used in conjunction.

The present work considers the application of Fourier transform near infrared (FTNIR) devices to noninvasive ethanol measurements. Several publications have discussed the underlying near infrared spectroscopic method (T. D. Ridder, S. P. Hendee, and C. D. Brown, “Noninvasive Alcohol Testing Using Diffuse Reflectance Near-Infrared Spectroscopy,” Applied Spectroscopy, 59(2), 181-189 (2005). T. D Ridder, C. D. Brown, and B. J. VerSteeg, “Framework for Multivariate Selectivity Analysis, Part II: Experimental Applications,” Applied Spectroscopy, 59(6), 804-815 (2005).) and its clinical comparison to blood and breath alcohol assays. T. Ridder, B. Ver Steeg, and B. Laaksonen, “Comparison of spectroscopically measured tissue alcohol concentration to blood and breath alcohol measurements,” Journal of Biomedical Optics, 14(5), (2009). T. Ridder, B. Ver Steeg, S. Vanslyke, and J. Way, “Noninvasive NIR Monitoring of Interstitial Ethanol Concentration,” Optical Diagnostics and Sensing IX, Proc. of SPIE Vol. 7186, 71860E1-11 (2009). T. D. Ridder, E. L. Hull, B. J. Ver Steeg, B. D. Laaksonen, “Comparison of spectroscopically measured finger and forearm tissue ethanol to blood and breath ethanol measurements,” Journal of Biomedical Optics, pp. 028003-1-028003-12, 16(2), 2011. The purpose of this work is to investigate an approach to calibration transfer that avoids methodologies that are commercially prohibitive due to the nature of noninvasive alcohol tests. For example, several commonly employed calibration transfer methodologies require a subset of the calibration samples to be measured on each device in order to determine a spectral transform that is applied to either the calibration data or to future validation data. Y. Wang, D. J. Veltkamp, and B. R. Kowalski, “Multivariate Instrument Standardization,” Anal. Chem., 63, 2750-2756, (1991). In the case of noninvasive alcohol testing, such approaches have limited applicability as obtaining ethanol containing spectra from humans on each instrument produced is cost prohibitive. Furthermore, it is unlikely that a subset of the human subject participants from the calibration study would be routinely available for measurement on devices produced in the future.

Instead, the calibration transfer approach of this work endeavors to develop a robust calibration that encompasses the range of instrument dependent spectral variation that would be encountered in present and future devices. The robust calibration is formed by combining clinically measured data acquired over a range of conditions with spectroscopic distortions derived from direct knowledge of the optical design of the spectrometer and the finite optical, mechanical, and alignment tolerances present in any practical instrument. The process is collectively referred to as Modification of Instrument Measurements by Incorporation of expert Knowledge (MIMIK). Thus, while the clinical calibration measurements are acquired from a small set of instruments, the MIMIK spectra encompass a larger range of inter-instrument variation. While the MIMIK spectra are amenable for use with traditional multivariate approaches such as PLS, PCR, and MLR at the time of initial calibration, they are also potentially suitable for use with methods such as PACLS and PACLS/PLS that seek to model sources of spectral variation not present in the original calibration data. C. M. Wehlburg, D. M. Haaland, D. K. Melgaard, and L. E. Martin, “New Hybrid Algorithm for Maintaining Multivariate Quantitative Calibrations of a Near-Infrared Spectrometer”, Applied Spectroscopy, 56(5), p. 605-614, 2002. D. K. Melgaard, D. M. Haaland, and C. M. Wehlburg, “Concentration Residual Augmented Classical Least Squares (CRACLS): A Multivariate Calibration Method with Advantages over Partial Least Squares”, Applied Spectroscopy, 56(5), p. 615-624, 2002. C. M. Wehlburg, D. M. Haaland, and D. K. Melgaard, “New Hybrid Algorithm for Transferring Multivariate Quantitative Calibrations of Intra-vendor Near-Infrared Spectrometers”, Applied Spectroscopy, 56(7), p. 877-886-614, 2002. In either case, the resulting multivariate calibration resulting from the MIMIK spectra is more robust to inter-instrument differences that might be encountered with future devices.

Ideal Interferometers and the Consequences of Finite Sized Light Sources

The noninvasive alcohol measurement system of the present work uses a Michelson geometry interferometer operating in the NIR (4000-8000 cm⁻) at 32 cm⁻¹ resolution. The interferometer, shown in FIG. 1, uses cube corner retroreflectors due to their reduced sensitivity to misalignment relative to flat mirrors. P. Griffiths, J. de Haseth, Fourier Transform Infrared Spectrometry, Wiley-Interscience, 1986. E. R. Peck, “Theory of the Corner-Cube Interferometer,” Journal of the Optical Society of America, pp. 1015-1024, 38(12), 1948. E. R. Peck, Uncompensated Corner-Reflector Interferometer, Journal of the Optical Society of America, pp. 250-252, 47(3), 1957. The purpose of the interferometer is to determine the spectrum associated with light introduced at its input and an ideal interferometer accomplishes this by modulating different wavelengths of light to different frequencies according to equation II-1.

F(x)=∫_{-∞}^{∞}B(σ)e^{i2πσx}dσ (II-1)

Where F(x) is the intensity measured at the detector as a function of optical path difference (x) and B(s) is the intensity of light at wavenumber s. F(x) is called the interferogram, the Fourier transform of which yields the desired intensity versus wavelength spectrum. Part 1 of this work demonstrated several optical effects encountered in practical instrumentation that violate the ideality assumptions implicit in equation II-1 and that a more applicable equation is:

F(x)=∫_{-∞}^{∞}B(σ)A(x,σ)e^{i(2πσx-φ(x,σ))}dσ (II-2)

Where A(x,σ) is a weighting surface that attenuates the interferogram intensity and φ(x,σ) is a surface that alters the phase of the interferogram. Both A(x,σ) and φ(x,σ) are functions of optical path difference and wavenumber and their specific forms depend on the types of non-idealities present in the interferometer under consideration. Part 1 of this description examined three sources of A(x,σ) and φ(x,σ) surfaces: self apodization due to beam divergence through the interferometer, misalignment (shear) of one or both retroreflectors relative to the optical axis, and off axis detector field of view (FOV).

The equations describing A(x,σ) and φ(x,σ) for self apodization are straightforward (additional discussions can be found elsewhere) (R. N. Feudale, N. A. Woody, H. W. Tan, A. J. Myles, S. D. Brown, J. Ferre, “Transfer of multivariate calibration models: a review,” Chemometrics and Intelligent Laboratory Systems, 64(2), p. 181-192, 2002. S. P. Davis, M. C. Abrams, J. W. Brault, Fourier Transform Spectrometry, Academic Press, 2001. J. Chamberlain, The Principles of Interferometric Spectroscopy, Wiley, 1979. G. A. Vanasse and H. Sakai, “Fourier Spectroscopy, Chapter 7”, Progress in Optics, vol 6, pp. 261-332, North-Holland Publishing Company, Amsterdam, 1967.):

$$\begin{matrix}A ( x , σ ) = sin c ( \frac{x σ}{2 π} Ω ) , \\ and & ( II \text{-} 3 ) \\ φ ( x , σ ) = σ x \frac{Ω}{2} , & ( II \text{-} 4 )\end{matrix}$$

where the solid angle is given by Ω=πρ₀² and ρ₀ is the divergence half angle of the collimated beam in radians. Examples of the impact of self apodization on the interferogram, instrument line shape, and spectra were provided in Part 1.

The functional forms of A(x,σ) and φ(x,σ) for retroreflector misalignment and off axis detector FOV are considerably more complex and have been described by Hearn and Murty. After considerable manipulation, Hearn and Murty arrive at the following equations for the weighting function (the solution to the integrals within Hearn are in terms of Lommel functions. There are two solutions, referred to as Un and Vn, only one of which is valid in a given situation. In Hearn's application one solution was valid at all evaluated points for the FT system under consideration. As a result, the second solution was not included. However, Murty shows both Lommel solutions as well as the means to determine which is valid for a given value of u and w (p and q in Murty). Murty also provides the reduced solution in the case that either u or w (p or q) is zero):

$$\begin{matrix}A ( x , σ ) = \frac{2}{u} \sqrt{{U}_{1}^{2} - {U}_{2}^{2}} for \vert \frac{u}{w} \vert \leq 1 , \\ and & ( II \text{-} 5 ) \\ A ( x , σ ) = \frac{2}{u} \sqrt{\begin{matrix}1 + {V}_{0}^{2} + {V}_{1}^{2} - \\ 2 {V}_{0} cos ( \frac{u}{2} + \frac{{w}^{2}}{2 u} ) - \\ 2 {V}_{1} sin ( \frac{u}{2} + \frac{{w}^{2}}{2 u} )\end{matrix}} for \vert \frac{u}{w} \vert > 1 , & ( II \text{-} 6 )\end{matrix}$$

And for the phase function:

$$\begin{matrix}φ ( x , σ ) = \frac{u}{2} - {tan}^{- 1} ( \frac{{U}_{2}}{{U}_{1}} ) for \vert \frac{u}{w} \vert \leq 1 , \\ and & ( II \text{-} 7 ) \\ φ ( x , σ ) = \frac{u}{2} + {tan}^{- 1} ( \frac{{V}_{0} + cos ( \frac{u}{2} + \frac{{w}^{2}}{2 u} )}{{V}_{1} - sin ( \frac{u}{2} + \frac{{w}^{2}}{2 u} )} ) for \vert \frac{u}{w} \vert > 1 , & ( 8 )\end{matrix}$$

Uₙ and Vₙ are the Lommel Functions defined as:

$$\begin{matrix}{U}_{n} = {\sum }_{i = 0}^{∞} {( - 1 )}^{i} {( \frac{u}{w} )}^{2 i + n} {J}_{2 i + n} ( w ) , \\ and & ( II \text{-} 9 ) \\ {V}_{n} = {\sum }_{i = 0}^{∞} {( - 1 )}^{i} {( \frac{w}{u} )}^{2 i + n} {J}_{2 i + n} ( w ) , & ( II \text{-} 10 )\end{matrix}$$

where n is the order of the Lommel function, i is the current term of the series expansion being computed, and J₂ᵢ₊ₙ is the Bessel function of order 2i+n. In general, we have found that three terms (max i of 2 in equations II-9 and II-10) is sufficient to calculate the weighting and phase functions with sufficient accuracy. A(x,σ) and φ(x,σ) can be determined using equations 5-10 for both shear and off axis detector FOV, albeit with a redefinition of u and w.

For Off-Axis Detector FOV:

u=2πσx cos(α₀)sin²(ρ₀), (II-11)

w=2πσx sin(α₀)sin(ρ₀), (II-12)

where α₀ is the angle of detector FOV misalignment in radians.

For Retroreflector Shear:

u=2πσx sin²(ρ₀), (II-13)

w=4πσs sin(ρ₀), (II-14)

where s is the retroreflector displacement from the optical axis in centimeters.

Part 1 of this description examined the A(x,σ) and φ(x,σ) surfaces corresponding to practical levels of misalignment of the associated optical components. Furthermore, Part 1 demonstrated laboratory methods for verifying the relevance of equations II-3 to II-14 as well as detecting the presence of their resulting distortions to the interferogram during interferometer alignment. In all cases, the effects of the A(x,σ) and φ(x,σ) surfaces yielded complex distortions to the instrument line shape in both the amplitude and wavenumber domains.

The calibration transfer approach of this work seeks to develop a robust calibration that incorporates the range of variation in the effects of self apodization, retroreflector misalignment (shear), and off axis detector FOV that might be encountered in a broad population of devices. The robust calibration is formed by modifying clinical in vivo data with the spectroscopic distortions described by equations II-3 to II-14. In order to lay the foundation for subsequent analyses, descriptions of the clinical study, FTNIR instrumentation, and data modification process are warranted.

### Experimental

Clinical Study Description

Alcohol excursions were induced in 108 subjects (demographics shown in Table 2) at Lovelace Scientific Resources (Albuquerque, N. Mex.) following overnight fasts. Written consent was obtained from each participant following explanation of the IRB-approved protocols (Quorum Review). Baseline venous blood and noninvasive NIR alcohol measurements were taken upon arrival in order to verify zero initial alcohol concentration. The alcohol dose for all subjects was ingested orally with a target peak blood alcohol concentration of 120 mg/dL (0.12%). The mass of the alcohol dose was calculated for each subject using an estimate of total body water based upon gender and body mass. An alcohol dose limit of 110 g was imposed to prevent overdosing obese subjects whose weight tended to overestimate their total body water.

TABLE 2
Patient demographics and environmental conditions from the clinical
study
Participant Demographics

| | Ethnicity | | | | | |
|---|---|---|---|---|---|---|
| | | Native | | Asian/ | | African |
| | Caucasian | American | Hispanic | Pacific Isl. | East Indian | American |
| # Subjects | 32 | 8 | 49 | 5 | 0 | 14 |

| | Age | | | | |
|---|---|---|---|---|---|
| | 21-30 | 31-40 | 41-50 | 51-60 | >60 |
| # Subjects | 33 | 16 | 26 | 26 | 7 |

BMI

| | 16-20 | 21-25 | 26-30 | 31-35 | 35-40 | >40 |
|---|---|---|---|---|---|---|
| # Subjects | 3 | 23 | 29 | 31 | 11 | 11 |

Gender

| | Male | Female |
|---|---|---|
| # Subjects | 50 | 58 |

Environmental Conditions

| | Min. | Max. |
|---|---|---|
| Temperature | 61° F. | 87° F. |
| Humidity | 15% | 78% |

Once the alcohol had been consumed and absorbed into the body, repeated cycles of venous blood and tissue alcohol measurements were acquired (˜25 minutes per cycle) from each subject until his or her blood alcohol concentration reached its peak and then declined below 20 mg/dL (0.02%). Under these conditions, the average alcohol excursion lasted approximately 7 hours. Ten noninvasive alcohol measurement devices of the same design participated in the study, with 6 of the 10 being used on any given day due to laboratory space limitations. Approximately 12 sets (minimum of 9 and maximum of 17) of tissue spectra and blood alcohol measurements were acquired per subject where each set contained 1 measurement from each of the 6 noninvasive instruments present on that day. Alcohol assays were performed on the blood samples using headspace gas chromatography (GC) analysis performed at Advanced Toxicology Network (Memphis, Tenn.). The ambient temperature and humidity of the clinical laboratory were orthogonally varied over the course of the study in order to maximize the range of environmental conditions captured by the study data (see Table 1 for the range of conditions spanned). A total of 7,661 sets of measurements were acquired from the 108 subjects.

Description of the FTNIR Alcohol Measurement

The noninvasive alcohol measurement employs NIR spectroscopy (4000 to 8000 cm⁻¹) which is of interest for noninvasive in vivo measurements because it offers specificity for a number of analytes, including alcohol and other organic molecules, while allowing optical path lengths of several millimeters through tissue, thus allowing penetration into the dermal tissue layer where alcohol is present in the interstitial fluid. G. L. Cote, “Innovative Non- or Minimally-Invasive Technologies for Monitoring Health and Nutritional Status in Mothers and Young Children,” Nutrition, 131, 1590S-1604S (2001). H. M. Heise, A. Bittner, and R. Marbach, “Near-infrared reflectance spectroscopy for non-invasive monitoring of metabolites,” Clinical Chemistry and Laboratory Medicine, 38, 137-45 (2000). V. V. Tuchin, Handbook of Optical Sensing of Glucose in Biological Fluids and Tissues, CRC press (2008). The noninvasive measurement devices were identical in design to those reported previously. T. D. Ridder, S. P. Hendee, and C. D. Brown, “Noninvasive Alcohol Testing Using Diffuse Reflectance Near-Infrared Spectroscopy,” Applied Spectroscopy, 59(2), 181-189 (2005). T. D Ridder, C. D. Brown, and B. J. VerSteeg, “Framework for Multivariate Selectivity Analysis, Part II: Experimental Applications,” Applied Spectroscopy, 59(6), 804-815 (2005). T. Ridder, B. Ver Steeg, and B. Laaksonen, “Comparison of spectroscopically measured tissue alcohol concentration to blood and breath alcohol measurements,” Journal of Biomedical Optics, 14(5), (2009). T. Ridder, B. Ver Steeg, S. Vanslyke, and J. Way, “Noninvasive NIR Monitoring of Interstitial Ethanol Concentration,” Optical Diagnostics and Sensing IX, Proc. of SPIE Vol. 7186, 71860E1-11 (2009). T. D. Ridder, E. L. Hull, B. J. Ver Steeg, B. D. Laaksonen, “Comparison of spectroscopically measured finger and forearm tissue ethanol to blood and breath ethanol measurements,” Journal of Biomedical Optics, pp. 028003-1-028003-12, 16(2), 2011. The interferometer (see FIG. 1) operated at 32 cm⁻¹ spectral resolution with a 0.8 cm/s scan speed which yielded 8-9 double sided, 2048 point interferograms per second. The spectral acquisition time was 1 minute for all measurements. The only requirement of the tissue measurements was passive contact between the tissue optical probe and the posterior surface of a finger at the medial phalange during the measurement period. The interferograms acquired during each 1 minute measurement were averaged and stored for subsequent use.

A spectroscopically and environmentally inert reflectance sample was measured on each instrument as a background at least every 20 minutes during the study by placing the reflectance sample over the optical probe surface. The measurement time of the reflectance sample was 1 minute and the resulting interferograms were averaged and stored. The most recent in time background interferogram from a given instrument was saved with each averaged in vivo interferogram for use in the interferogram modification process as well as background correction during subsequent spectral processing. The experimental data were imported into Matlab 2012a, which was used to perform all data processing and analyses.

Modification of Clinical Data with the Derived Weighting and Phase Functions

A significant challenge of calibration transfer is that data collected from a single instrument, or limited number of instruments, does not adequately represent data from future instruments. Several equations have been shown that describe important sources of spectral distortion arising from realistic variation in the alignment of optical components within devices employing an interferometer. It is important to note that, despite best efforts, each instrument produced certainly contains an unknown amount of misalignment in every component. As a result, the objective of the present work is to modify experimentally collected data from a set of instruments with physically appropriate relative weight, A(x,σ), and phase, φ(x,σ), surfaces using the equations 3-14. Relative measures are used because experimentally acquired, rather than ideal, interferograms are being modified. The experimental interferograms already inherently contain unknown amounts of spectral distortions caused by self apodization, shear, and detector alignment. As such, the MIMIK process seeks to modify already non-ideal experimentally acquired interferograms to be further non-ideal in ways that are likely to be encountered with future instruments.

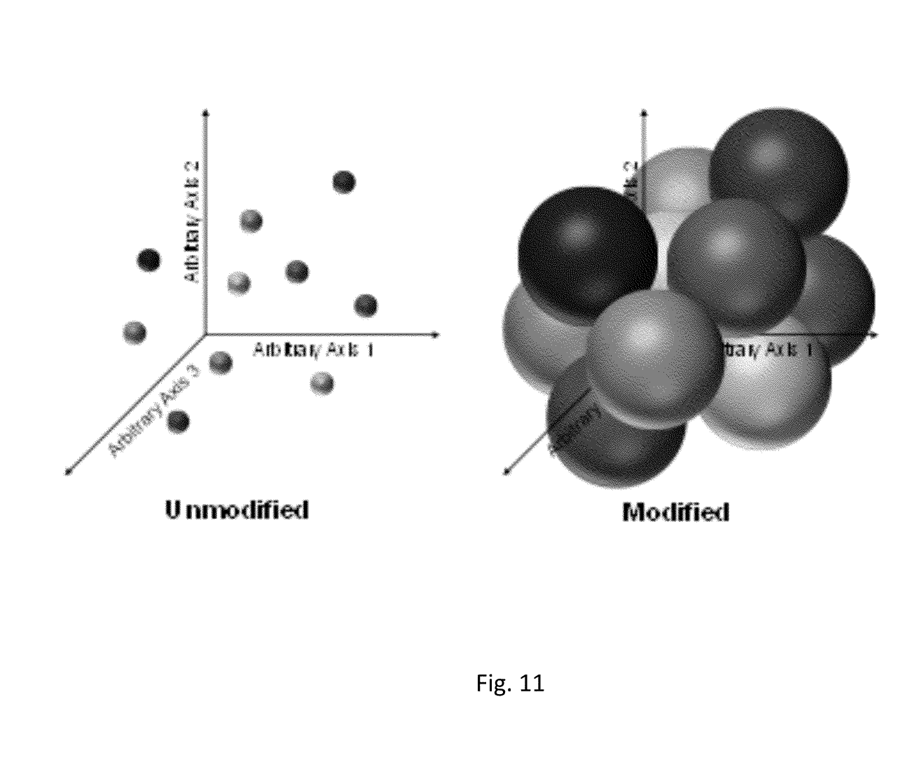

The MIMIK approach is shown pictorially in FIG. 11. The left window of FIG. 11 depicts an arbitrary space populated by 10 spheres, each of which represents data acquired from a single instrument. The general premise is that the arbitrary coordinate system adequately explains the data from each instrument, but the fact that they each reside in a unique location in the arbitrary space results in a calibration transfer challenge. It should be noted that there may also be instrument specific covariance in the arbitrary space that is not shown by the perfect spheres in the pictorial shown in FIG. 11 that can also impact calibration transfer. In any case, the objective is to reduce the magnitude of the calibration challenge by intentionally growing, and more densely covering, the arbitrary space to increase the likelihood that data acquired from future instruments will be encompassed by the space spanned by the resulting MIMIK calibration data (pictorially shown right window of FIG. 11).

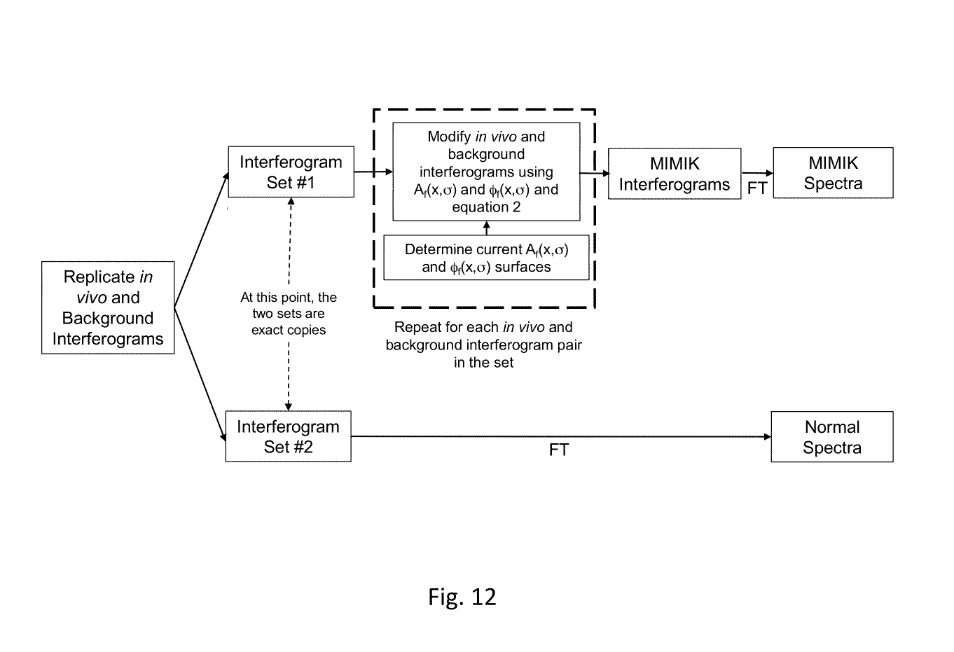

The MIMIK process (see FIG. 12) begins by replicating the clinical data (in vivo and background interferograms) into two identical sets, each containing all of the measured interferograms. One set of interferograms is left “as is” and converted to “normal” spectra via Fourier transform. In the second set, each pair of in vivo and reflectance background interferograms is modified by weighting, A(x,σ), and phase, φ(x,σ), surfaces according to equation 2. The process is repeated until all pairs of in vivo and background interferograms in the set have been modified. The resulting interferograms are then Fourier transformed to form a set of “MIMIK” spectra.

The steps by which A(x,σ) and φ(x,σ) are determined as well as the modification process for a given pair of in vivo and reflectance background interferograms, collectively shown in FIG. 12 as the dashed box, are important aspects of the present work and are discussed in more detail below. Table 3 shows the parameters, descriptions, and ranges of values used during the modification process.

TABLE 3
Ranges and description of parameters used to determine A(x, σ) and φ(x, σ)

| Parameter | Description | Minimum | Maximum | Purpose |
|---|---|---|---|---|
| ζ_{0.4000} | Angular divergence | 2.7 | 4.5 | Simulates poor collimating |
| | of the collimated | | | lens alignment |
| | beam at 4000 cm⁻¹ | | | |
| dζ₀/dζ | Linear dependence of | −1.2 5 × 10⁻⁴/ζ | +1.25 × 10⁻⁴/ζ | Simulates poor collimating |
| | ζ₀ on wavenumber | | | lens alignment, chromatic |
| | | | | aberration |
| S₁ | Shear at minimum | −4 mm | +4 mm | Retroreflector |
| | OPD (x) | | | misalignment, off axis |
| | | | | retroreflecter trajectory |
| S₂ | Shear at maximum | −4 mm | +4 mm | Retroreflecter |
| | OPD (x) | | | misalignment, off axis |
| | | | | retroreflecter trajectory |
| ζ₀ | Angle of detector | 0 | 1 | Misalignment ot the |
| | FOV displacement | | | detector FOV |
| | from the optical axis | | | |

Description of the MIMIK Steps

Step 1:

Calculate Aₚ(x,σ) and φₚ(x,σ) using equations 3 and 4 using a constant angular divergence, ρ₀, of 3.6 degrees. The subscript, p, denotes “perfect” and is indicative of the weight and phase surfaces that would result from a perfectly aligned interferometer (no shear or off-axis detector FOV) with self apodization caused by a 3.6 degree diverging beam at all wavenumbers. These perfect surfaces are used in the modification of all interferograms in the set, and as such only need to be determined once. Subsequent steps assume a single pair of in vivo and background interferograms and that steps 2-13 are repeated for each pair of interferograms in the set.

Step 2:

Randomly draw values for the divergence half angle of the collimated beam at 4000 cm⁻¹, ρ_{0,4000}, and its linear wavenumber dependence, dρ₀/dσ, from uniform distributions in the ranges shown in Table 2. Use ρ_{0,4000}, dρ₀/dσ, and the equation of a line to determine ρ₀ for all σ, referred to as ρ_{0,σ}.

Step 3:

Randomly draw a value for the misalignment of the detector FOV, α₀, from a uniform distribution in the range specified in Table 3.

Step 4:

Use α₀ and ρ_{0,σ} and equations II-5 to II-10 and the definitions of u and w for off axis detector FOV (equations 11 and 12) to calculate A_{d}(x,σ) and φ_{d}(x,σ). For purposes of differentiation from surfaces in other steps the subscript, d, denotes “detector”. It is important to note that A_{d}(x,σ) and φ_{d}(x,σ) inherently contain the effects of both wavenumber dependent self apodization (ρ_{0,σ} varies with wavenumber) and off-axis detector FOV.

Step 5:

A_{d}(x,σ) and φ_{d}(x,σ) from step 4 would be applicable to the modification of an ideal interferogram from an interferometer with perfect collimation and perfect alignment. The resulting interferogram would appear to have come from an interferometer with beam divergence specified by ρ_{0,σ} and detector FOV misalignment specified by α₀. However, the clinically acquired interferograms to be modified are not ideal and already inherently contain the effects of self apodization and detector FOV alignment to an unknown extent. Consequently, direct application of A_{d}(x,σ) and φ_{d}(x,σ) to the clinical interferograms would result in excessive spectral distortions not representative of other instruments of the same design. As such, A_{d}(x,σ) and φ_{d}(x,σ) are made relative by element wise operations according to:

A_{d}(x,σ)=A_{d}(x,σ)/Aₚ(x,σ), (II-17)

and

φ_{d}(x,σ)=φ_{d}(x,σ)−φₚ(x,σ), (II-18)

The resulting A_{d}(x,σ) and φ_{d}(x,σ) surfaces represent the deviations in weight and phase, respectively, from the perfectly aligned, constant divergence angle case determined in step 1.

Step 6:

Randomly draw values for shear at the limits of OPD, s₁ and s₂, using uniform distributions over the ranges specified in Table 2. Calculate s as a function of OPD, sₓ, using s₁ and s₂ and the equation of a line.

Step 7:

Use sₓ and ρ_{0,σ} with equations 5-10 and the definitions for u and w for retroreflector shear (equations II-13 and II-14) to calculate Aₛ(x,σ) and φₛ(x,σ) where the subscript, s, denotes “shear”. Similar to the surfaces calculated in step 4, Aₛ(x,σ) and φₛ(x,σ) contain the effects of both wavenumber dependent self apodization and OPD dependent shear (sₓ) which would be suited to modify an ideal interferogram. Consequently, Aₛ(x,σ) and φₛ(x,σ) need to be made relative such that they are applicable to the modification of the clinical interferograms. However, A_{d}(x,σ) and φ_{d}(x,σ) from step 5 already contain the effects of wavenumber dependent self apodization caused by ρ_{0,σ} relative to the constant ρ₀ surfaces, Aₚ(x,σ) and φₚ(x,σ), from step 1. Aₛ(x,σ) and φₛ(x,σ) also contain the effects of wavenumber dependent self apodization. As such, an extra step needs to be performed in order to prevent it from being erroneously included in the modification process twice.

Step 8:

Use ρ_{0,σ} with equations 3 and 4 to calculate A_{σ}(x,σ) and φ_{σ}(x,σ), which contain the effects of wavenumber dependent self apodization for an interferometer with no shear (s=0) or detector FOV misalignment (α₀₌₀). Remove the effects of wavenumber dependent self apodization from Aₛ(x,σ) and φₛ(x,σ) using:

Aₛ(x,σ)=Aₛ(x,σ)/A_{σ}(x,σ), (II-19)

and

φₛ(x,σ)=φₛ(x,σ)−φ_{σ}(x,σ), (II-20)

Note that A_{σ}(x,σ) and φ_{σ}(x,σ) from this step and Aₚ(x,σ) and φₚ(x,σ) from step 1 are not identical as A_{σ}(x,σ) and φ_{σ}(x,σ) are dependent on ρ_{0,σ} from step 2. As a result, A_{σ}(x,σ) and φ_{σ}(x,σ) must be calculated whenever ρ_{0,σ} changes.

Step 9:

Combine the surfaces from steps 5 and 8 using:

A_{f}(x,σ)=Aₛ(x,σ)A_{d}(x,σ), (II-21)

and

φ_{f}(x,σ)=φₛ(x,σ)+φ_{d}(x,σ), (II-22)

Where the subscript, f, denotes “final”.

Step 10:

Obtain B(σ) for the background interferogram via Fourier transform using the Mertz method. The Mertz method is used to obtain B(σ) because it also yields an estimate of the optical phase function, φ_{Opt}. The optical phase function explains sources of dispersion differences between the two legs of the interferometer, such as a mismatch in the thickness of the beam splitter and compensating plate (see FIG. 1), and is distinct in origin and manifestation from the phase distortions caused by self apodization, shear, and off-axis detectors. It is assumed for this work that φ_{Opt} varies with wavenumber but is constant for all OPD's. φ_{f}(x,σ) is modified to account for the optical phase by adding φ_{Opt} to each column.

Step 11:

Use A_{f}(x,σ), φ_{f}(x,σ), and B(σ) in conjunction with equation II-2 to calculate a 1^{st} MIMIK background interferogram. Use 1/A_{f}(x,σ), −φ_{f}(x,σ), and B(σ) in conjunction with equation II-2 to calculate a 2^{nd} MIMIK background interferogram.

Step 12:

Obtain B(σ) for the in vivo interferogram paired with the background interferogram via Fourier transform. Use A_{f}(x,σ), φ_{f}(x,σ), and B(σ) in conjunction with equation II-2 to calculate a 1^{st} MIMIK in vivo interferogram. Use 1/A_{f}(x,σ), −φ_{f}(x,σ), and B(σ) in conjunction with equation II-2 to calculate a 2^{nd} MIMIK in vivo interferogram.

Step 13:

Fourier transform the MIMIK background and in vivo interferograms from steps 11 and 12 and store the resulting spectra in the “MIMIK” spectral set.

Step 14:

Repeat steps 2-13 for all in vivo, background interferogram pairs.

One way to think of the use of B(σ) obtained from the transform of the experimental interferogram is that it is already impacted by self apodization, shear, and off axis detector FOV, but to an unknown extent. Referring back to FIG. 11, the objective of the modification approach is to expand the space spanned by each instrument in order to fill out the space that describes inter-instrument differences. By making A_{f} (x,σ) and φ_{f}(x,σ) relative to a perfectly aligned interferometer, subsequent application to experimental interferograms imparts a distortion that is relative to the unknown instrument centers from which the interferograms were taken. Furthermore, for a given clinical interferogram, the application of A_{f}(x,σ) and φ_{f}(x,σ) expands the arbitrary space in on direction relative to the unknown center while the application of 1/A_{f}(x,σ) and −φ_{f}(x,σ) expands it in the opposite direction. Thus, referring back to FIG. 11, the use of 1/A_{f}(x,σ) and −φ_{f}(x,σ) in addition to A_{f}(x,σ) and φ_{f}(x,σ) increases the span of each sphere in both directions of each arbitrary axis rather than just in one direction if only A_{f}(x,σ) and φ_{f}(x,σ) were used.

Results and Discussion

Spectral Comparison

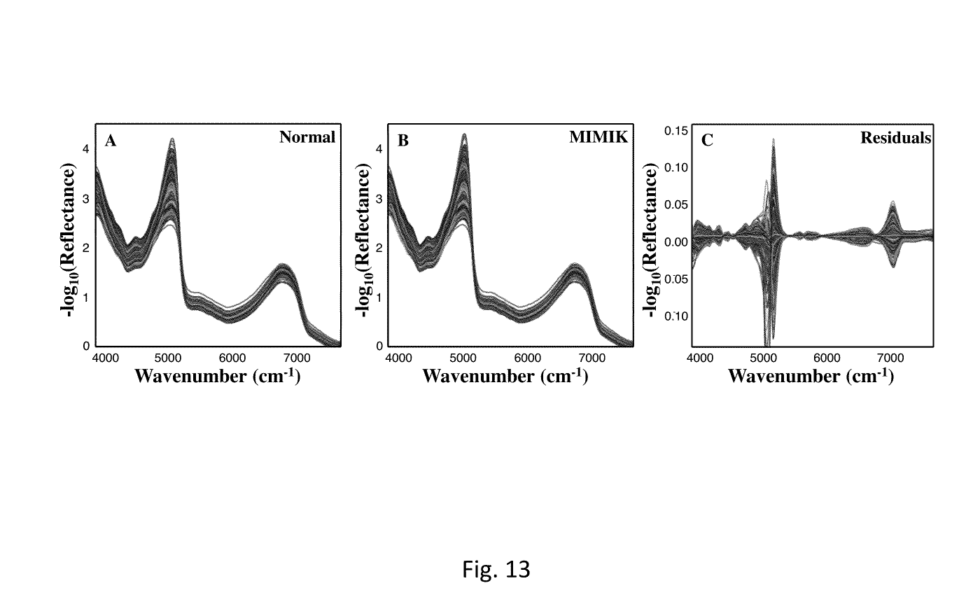

Part 1 of this description showed the effects of the distortions in terms of line shape and wavenumber shift. As the purpose of this work is to examine their influence on calibration transfer it is important to examine the spectral distortions caused by self apodization, shear, and off axis detector FOV for the in vivo data. FIG. 13 shows the background corrected normal spectra (Window A), background corrected MIMIK spectra (Window B), and their difference (Window C). Note that while the MIMIK spectral set is twice as large as the normal set because each interferogram was modified once by A_{f}(x,σ) and φ_{f}(x,σ) and once by 1/A_{f}(x,σ) and −φ_{f}(x,σ), the normal and MIMIK spectra are otherwise identical in the sense that they originate from the same patients, instruments, and study conditions.

The residuals of both sets of MIMIK (A_{f}(x,σ), φ_{f}(x,σ) and 1/A_{f}(x,σ), −φ_{f}(x,σ)) and the normal spectra are shown in Window C of FIG. 13. For perspective, the absorbance spectrum of 80 mg/dL ethanol measured in transmission using a 1 mm path length has a maximum value of 0.002 A.U. No attempt is being made to suggest that the transmission spectrum of ethanol in any way represents the in vivo ethanol signal measured in reflectance. However, the comparison is useful in the sense that the magnitude of spectral residuals is certainly large enough that the distortions caused by self apodization, shear, and detector FOV alignment are worthy of attention.

Calibration/Validation Cases Tested

Examination of the effect of background correction is important in the context of this work as it is often used as a means for compensating for several types of instrument effects. However, the benefits of background collection are limited to multiplicative effects in the intensity domain such as light source intensity, light source color temperature, and detector response. Background correction has no impact on spectral distortions such as lineshape changes or wavelength shifts. In contrast, while the modification process of the present work does address some effects that are multiplicative in intensity, it also seeks to address the physical phenomena that result in spectral convolutions and wavenumber shifts. Thus, it is surmised that background correction and the modification process of the present work likely address different sources of inter-instrument spectral variation and it is important to examine their independent and cumulative effects.

Towards that end, Table 4 shows the four cases of calibration data tested. The validation set is normal for all cases as that reflects the type of data that would be prospectively collected on future instruments. Furthermore, no outliers were removed from the validation set in any of the cases. Thus, the number of measurements, as well as their origins (e.g. patient, instrument, day, etc.), were identical in all four cases examined.

TABLE 4
Calibration/Validation cases tested

| | Calibration | Validation | Background |
|---|---|---|---|
| | Type | Type | Correction |
| Case A | Normal | Normal | No |
| Case B | Normal | Normal | Yes |
| Case C | MIMIK | Normal | No |
| Case D | MIMIK | Normal | Yes |

Cross Validation Approach

Cross validation was used to examine the effects of the MIMIK process and background correction on the calibration transfer of the noninvasive ethanol measurements. The objective of the cross validation analysis is to attempt to assess the robustness of the multivariate ethanol regression to spectra acquired from new people on new instruments as would be encountered as instruments are deployed. Random leave-N-out or similar cross validation schemes are not particularly useful towards performing that assessment because spectral information from a given subject and/or instrument in the held-out set can remain in the calibration set.

Instead, a subject/instrument-out cross validation approach was used in this work and is described as follows.

1) All subject-instrument combinations were identified in the validation set.
2) The validation measurements from a single subject-instrument combination were “held-out” for subsequent prediction.
3) All data from the person in step 2 on all instruments was removed from the calibration set.
4) All data from the instrument in step 2 from all people was removed from the calibration set.
5) Partial Least Squares (PLS) was used in conjunction with the remaining calibration spectra to obtain an ethanol regression model.
6) The held out data from step 2 was predicted and associated Mahalanobis distance and spectral F-ratio metrics were determined.
7) The removed calibration data is returned to the set.
8) Steps 2-7 are repeated until all validation subject-instrument combinations have been evaluated.

Results from Cross Validated PLS

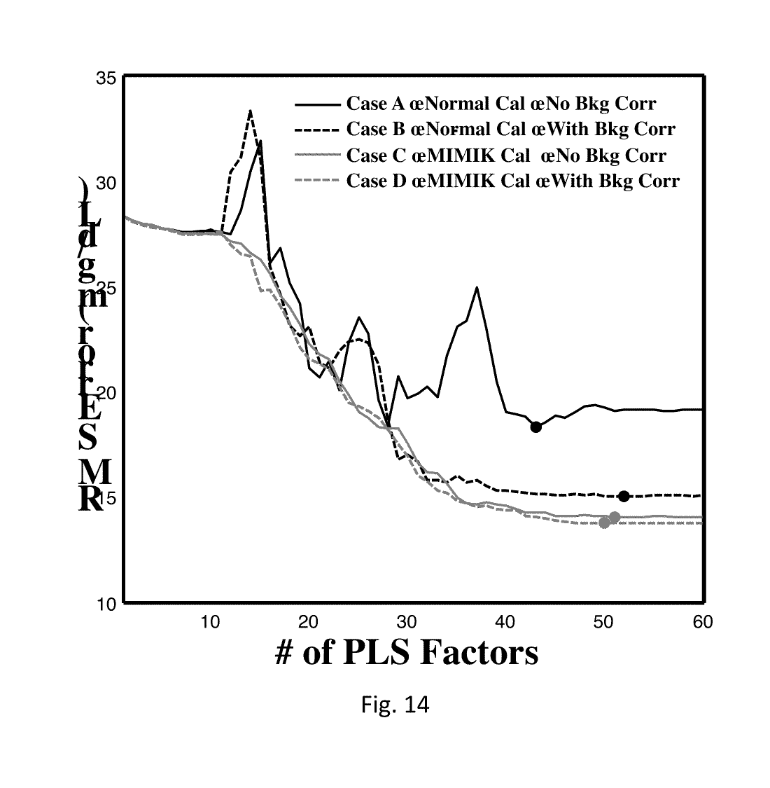

FIG. 14 shows the root mean squared error of cross validation (RMSECV) obtained from Partial Least Squares (PLS) regression for the four cases. Additional information regarding PLS regression can be found elsewhere. The solid dot on each RMSECV curve corresponds to the optimum number of factors for that case determined using Akaike's Information Criterion (AIC). H. Akaike, IEEE Trans. Automat. Control 19, 716 (1974). The objective of determining the optimum number of factors for each case is to allow comparison of outlier metric behavior between the four cases.

The RMSECV obtained from the normal calibration data with no background correction (Case A) is denoted by the solid black line in FIG. 14. While the RMSECV curve does exhibit some degree of convergence (more factors generally decrease error), it has a poor shape and erratic behavior with some later factors significantly inflating prediction error. It might be hypothesized that the shape and behavior can be attributed to an insufficient amount of calibration data. However, all CV iterations for each case had greater than 6,000 spectra during the formation of regression model. Simply increasing the quantity of data would therefore not be expected to alter the behavior of the normal, no background RMSECV curve. This suggests that, for this case, there are spectroscopic variations in the validation set that are problematic for the multivariate regression when neither background correction nor MIMIK is applied.

Case B is denoted by the black dashed line in FIG. 14 and represents the case where the calibration set was normal and background correction was employed. Clearly, background correction offers significant improvement relative to the no background correction case (15.0 mg/dL at 52 factors versus 18.2 mg/dL at 43 factors). This suggests that one or more sources of multiplicative intensity variation are present in the data and that background correction is a useful form of compensation. Case C (MIMIK calibration set with no background correction) is represented by the grey solid line in FIG. 5 which demonstrates that the MIMIK method is effective at reducing error in the validation set (14.0 mg/dL at 51 factors) relative to both of the normal calibration set cases (A and B). Furthermore, the resulting RMSECV curve exhibits a more smooth progression in error with each added factor. Combination of the MIMIK data with background correction (Case D, dashed grey line in FIG. 14) results in the best overall performance of 13.7 mg/dL at 50 factors.

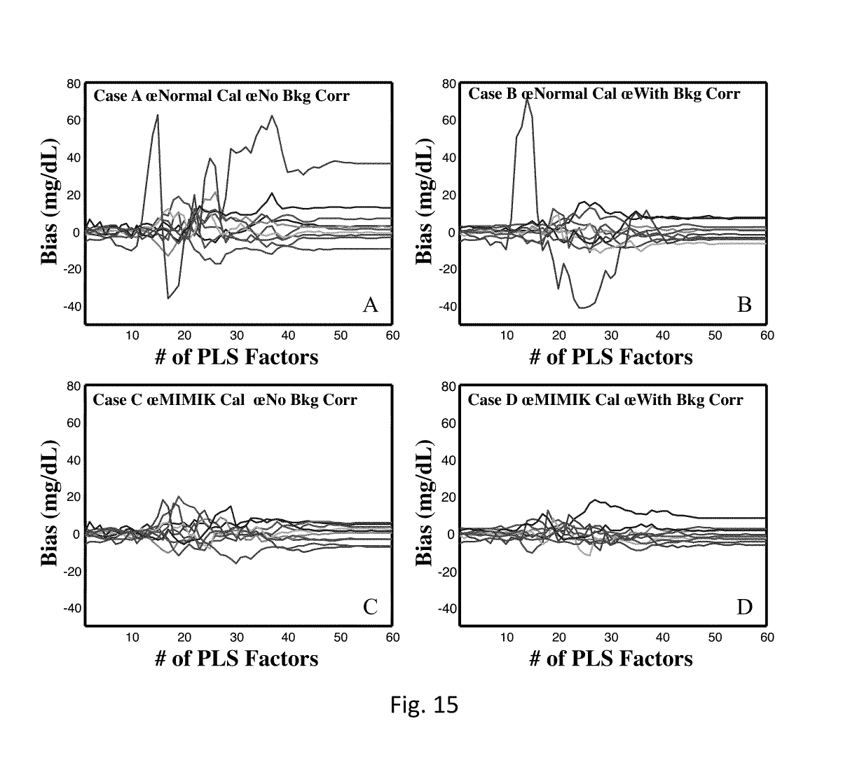

It is surmised that the differences in RMSECV's in FIG. 14 are related to inter-instrument variation. FIG. 15 shows the bias by factor for each of the 10 instruments that participated in the study. The normal calibration with no background correction case (Window A) exhibits several instruments with significant biases that also vary strongly as a function of the number PLS factors. For example, the solid black curve shows that its associated instrument has a 30.1 mg/dL prediction bias at 43 factors, which is the optimum for this case as determined by AIC. In short, the ethanol regression model formed from the data obtained from the other 9 does not generalize well to the spectral measurement obtained from this particular instrument. Examination of the Window A of FIG. 15 indicates that there are other instruments in the set that also exhibit various degrees of prediction bias variation across the range of factors tested.

Window B of FIG. 15 shows the instrument biases as a function of PLS factors for the validation predictions obtained from the normal calibration set with background correction. Comparison of Windows A and B shows that background correction is certainly beneficial, particularly at factors greater than 40. However, at lower factors, several instruments still exhibit significant structure in their bias curves. A possible explanation for this phenomenon is that the corresponding factors could be related to spectral distortions that arise from, or are sensitive to, changes in lineshape or wavenumber shifts that background correction would be unable to address.

Window C of FIG. 15 shows the impact of the MIMIK calibration data on validation instrument bias when no background correction is performed. At high factors, the overall impact is similar to that of background correction. However, at lower factors the behavior is generally improved, particularly the instrument denoted by the black trace. This could indicate that the MIMIK data results in a more generalized ethanol regression that is less sensitive to inter instrument variations in the associated model factors. Similar to the RMSECV curves shown in FIG. 14, the combination of modification and background correction (Window D of FIG. 15) not only offers the best overall suppression of instrument bias at higher factors where predictions would presumably be made, but also the most stable bias behavior at all factors. This is an indicator that while some overlap in the effects of modification and background correction likely exists, they each address spectral phenomena the other cannot.

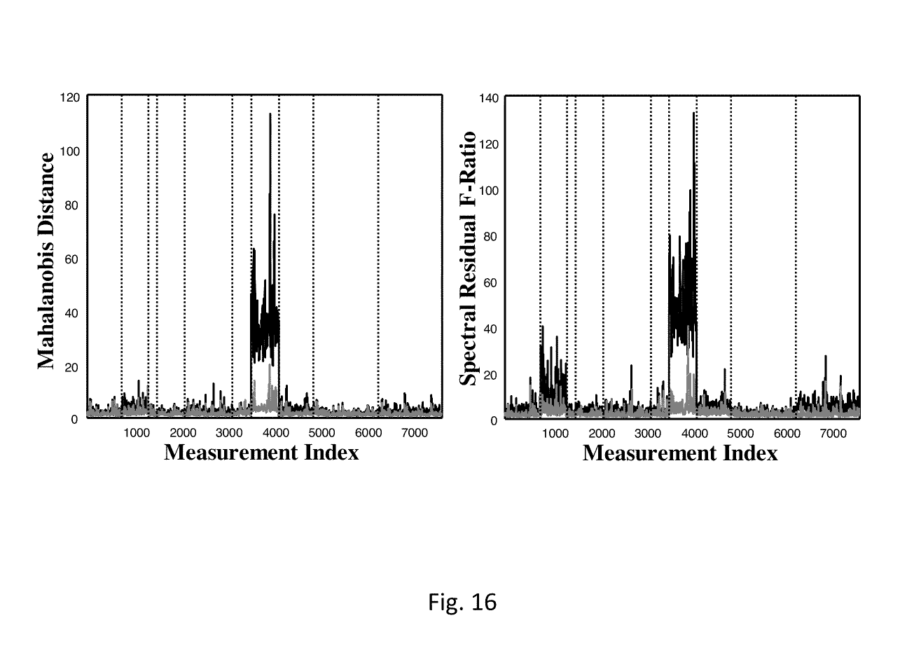

In addition to measurement error, the robustness of the regression model to future data is also an important consideration. Outlier metrics such as the Mahalanobis distance and the spectral F-ratio are useful in determining the consistency of data to be predicted with the data used to form the regression model. Towards that end, FIG. 16 compares the Mahalanobis distance and spectral F-ratio metrics obtained for the validation data for the normal calibration data, no background correction case (Case A, solid black line) and the MIMIK calibration data with background correction case (Case D, solid grey line). The metrics are sorted by instrument and the dotted, vertical lines in FIG. 16 indicate transitions between instruments. Examination of the Case A metrics shows that several instruments exhibit inflated Mahalanobis distances and spectral F-ratio's. It is worthy to note that the instrument with the large bias in metric values for is also the instrument with the large prediction biases shown in the Windows A and B of FIG. 15.

The grey line shows the validation metric values corresponding to Case D. Several of the instruments exhibit significantly reduced biases in their metric values. Table 5 shows the median Mahalanobis and spectral F-ratios by instrument for the four cases tested. It is important to note that the smaller values for the outlier metrics in the MIMIK, background corrected case are not indicative that the validation spectra have been moved or corrected towards the center of the calibration. Instead, the calibration space has been intentionally grown such that the validation spectra are closer to the center of the calibration space in a relative sense. In any case, the metric values shown in FIG. 16 and Table 5 indicate that the MIMIK process has a significant effect on improving the robustness of the ethanol regression model to data acquired from future instruments.

TABLE 5
Median metric values by instrument for cases A-D

| | Inst. 1 | Inst. 2 | Inst. 3 | Inst. 4 | Inst. 5 | Inst. 6 | Inst. 7 | Inst. 8 | Inst. 9 | Inst. 10 |
|---|---|---|---|---|---|---|---|---|---|---|
| Median Mahalanobis | | | | | | | | | | |
| Distance | | | | | | | | | | |
| Normal Cal No Bkg | 1.8 | 3.6 | 1.6 | 1.7 | 2.2 | 1.9 | 34.0 | 2.9 | 1.5 | 2.3 |
| Normal Cal With Bkg | 1.3 | 3.7 | 1.3 | 1.4 | 1.3 | 1.8 | 20.1 | 2.4 | 1.4 | 1.7 |
| MIMIK Cal No Bkg | 1.2 | 1.7 | 1.3 | 1.3 | 1.3 | 1.6 | 3.5 | 1.7 | 1.3 | 1.4 |
| MIMIK Cal With Bkg | 1.1 | 1.7 | 1.1 | 1.1 | 1.0 | 1.4 | 3.4 | 1.3 | 1.0 | 1.0 |
| Median Spectral | | | | | | | | | | |
| Residual F-Ratio | | | | | | | | | | |
| Normal Cal No Bkg | 3.2 | 9.6 | 2.2 | 3.7 | 2.7 | 2.3 | 46.1 | 4.6 | 2.3 | 4.9 |
| Normal Cal With Bkg | 2.0 | 5.4 | 1.5 | 1.9 | 1.7 | 2.0 | 25.9 | 4.1 | 1.6 | 3.4 |
| MIMIK Cal No Bkg | 1.6 | 3.2 | 1.7 | 1.9 | 1.5 | 1.6 | 5.6 | 2.2 | 1.7 | 2.0 |
| MIMIK Cal With Bkg | 1.3 | 3.2 | 1.4 | 1.6 | 1.3 | 1.6 | 4.1 | 2.3 | 1.5 | 1.5 |

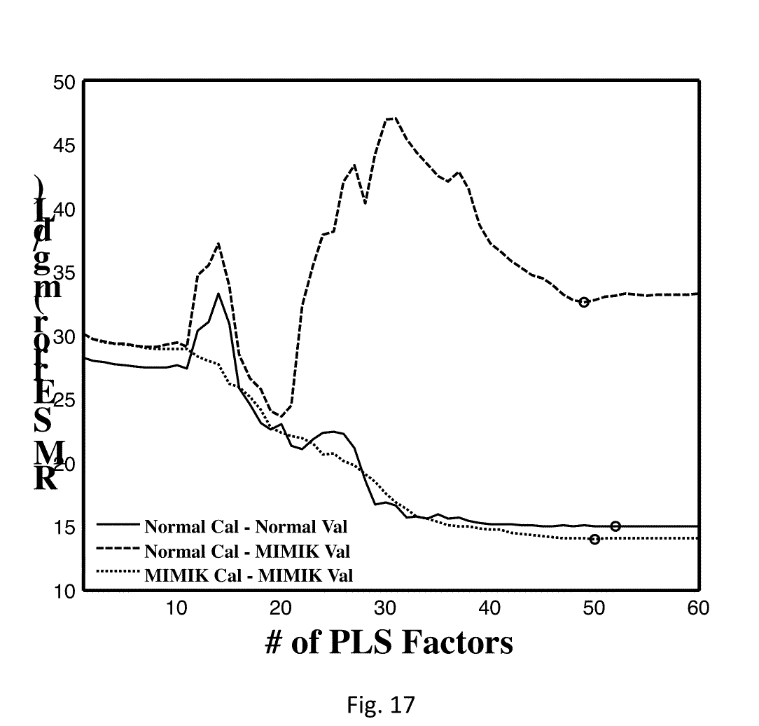

An alternative perspective of the effects of self apodization, shear, and off axis detector FOV was obtained by examining the predictions of the MIMIK data. FIG. 17 shows the RMSECV curves obtained for three cases, all of which employed background correction: the normal data as both the calibration and validation set, the normal data predicting the MIMIK data, and the MIMIK data as both the calibration and validation set. Absent any knowledge or concerns regarding the optical distortions presented in this work, the RMSECV for the normal data predicting itself (solid line in FIG. 17) could be thought of as a nave performance estimate for alcohol measurements obtained from a broad population of future instruments. However, if the true variation in alignment in future instruments spans the ranges shown in Table 3, the solid line in FIG. 17 would be an optimistic view of alcohol measurement accuracy. Instead, the dashed line (normal data predicting the MIMIK data) would be a better estimate of validation error in this scenario.

Clearly, the difference in the solid and dashed lines in FIG. 17 is substantial enough to suggest that the distortions presented to this work must be constrained in terms of magnitude via tighter alignment tolerances than those shown in Table 3, accommodated in the multivariate regression, or a combination of both. While the Cases C and D in FIG. 14 indicate that inclusion of the spectral distortions in the calibration set via the MIMIK process is beneficial to alcohol measurement accuracy, a fair question is to ask how well the MIMIK data predicts itself. The dotted line in FIG. 17 shows the RMSECV curve for this case. Interestingly, the MIMIK data predicting itself yields and RMSECV of 14.0 mg/dL at 50 factors which compares very well to the RMSECV observed for the MIMIK data predicting the normal data (13.7 mg/dL at 50 factors, see FIG. 14). The similarity of the RMSECV's suggests that incorporation of the spectral distortions caused by self apodization, shear, and off axis detector FOV can be accommodated by the multivariate regression without substantial degradation of the net analyte signal.

Part 1 of this description showed that the equations describing spectral distortions in FTNIR could be observed in laboratory measurements and that the distortions were complex in both the intensity and wavenumber domains. Part 1 also showed that laboratory measurements could be incorporated into the interferometer alignment process in order to reduce inter-instrument variation as well as identify problematic optical alignment tolerances which in turn could be used to refine the interferometer design. Part 2 explicitly incorporates knowledge of the manifestations of the distortions into the calibration data in order to improve the generalization of the multivariate regression to measurements performed on future instruments. The analysis of the normal and MIMIK data sets showed that some differences observed between instruments are indeed related to the self apodization, retroreflector misalignment, and off axis detector FOV and that the presented equations were useful in synthetically incorporating their effects into the calibration data. The inclusion of the spectral distortions in the MIMIK calibration data significantly reduced the noninvasive ethanol measurement error while also yielding outlier metric values that suggested the multivariate regression was less sensitive to inter-instrument differences.

While the focus of this description was FTNIR measurements of in vivo ethanol, the MIMIK approach can be extended to other applications as well as instrument designs other than the cube corner interferometer design used in this work. Part 1 of this description identified several areas within a Michelson interferometer where practice departs from the ideal theory presented in many texts and that those departures yield wavenumber dependent distortions to the instrument line shape and wavenumber axis. It is important to note that other spectrometer designs, whether interferometric or dispersive, certainly have similar dependencies on practical optics and alignment tolerances. While the signal measured by any spectrometer is a function of the intensity versus wavelength of the light at its input, it is also dependent on several other parameters including the range of angles propagating through the spectrometer. As collimation is never perfect, it follows that all spectrometer types yield spectra that depend on optical components that alter angular content as well as their individual and relative alignment. It is up to the practitioner to determine which optical parameters are important to their particular application and spectrometer.

One area of future expansion contemplated by the present invention is to perform an analysis of variance to determine which types of distortion are the most problematic to the noninvasive ethanol measurement. Cross terms of the analysis can also be examined in order to determine if the simultaneous presence of different distortions yields larger measurement errors. The behavior of the RMSECV curves shown in FIGS. 14 and 17 suggests that each PLS factor is impacted by the distortions to a different degree. Decomposing the RMSECV curves into individual distortions could offer insights into the types of spectral effects the corresponding factors are attempting to accommodate.

Another area of future interest is to determine if the equations presented in this work can be expanded to accommodate interactions between the samples (the patients in this work) and the instrument. For example, scattering samples such as tissue often impart angular and spatial structure to the light introduced to the spectrometer. If the imparted angular and spatial structure interacts with the collimating lens of the interferometer such that the angular divergence of the collimated beam is altered, there would in turn be an interaction with the effects of self apodization, retroreflector misalignment, and off axis detector FOV. If it were determined that the spectral manifestation of the interactions were important to the multivariate regression, it would follow that the modification process applied in this work could be adapted to accommodate the sample dependent effects.

The method described in this work seeks to develop a multivariate regression that includes the range of optical distortions expected in future instruments. An alternative approach is to actively correct incoming measurements for their specific distortions using empirically derived weight, A_{f}(x,σ), and phase, φ_{f}(x,σ), surfaces. In other words, rather than growing the calibration space to encompass future data, the future data would be corrected such that it was closer to the center of the calibration space. Indeed, both methods could be employed simultaneously in order to help ensure future data falls within the calibration space.

One consideration of the correction approach is that equations II-2 to II-14 would need to be actively evaluated in order to determine the appropriate correction surfaces for a given measurement. A fitness function such Mahalanobis distance can be used to determine when the applied surfaces have appropriately shifted the measurement within the calibration space. However, evaluation of equations 2-14 involve several integrations and their subsequent application requires multiple Fourier transforms. Thus, active correction approaches should consider computational requirements, particularly if real time or near real time results are required.

Those skilled in the art will recognize that the present invention can be manifested in a variety of forms other than the specific embodiments described and contemplated herein. Accordingly, departures in form and detail can be made without departing from the scope and spirit of the present invention as described in the appended claims.

We claim:

1. A method of producing a plurality of spectroscopic measurement devices, comprising:
(a) producing a calibration model that includes the expected range of measurement variation across the plurality of devices;
(b) producing the devices;
(c) installing the calibration model on each device.

2. A method as in claim 1, further comprising determining the expected range of measurement variation from an analytical model of the device.

3. A method as in claim 1, wherein producing a calibration model comprises: collecting one or more base calibration spectra on a base instrument; producing a plurality of synthetic calibration spectra from the base calibration spectra with a transfer function determined from the device design; and producing the calibration model from the base calibration spectra and the synthetic calibration spectra.

4. A method as in claim 1, wherein the spectroscopic measurement device is one or more of: a Fourier transform spectrometer, a dispersive spectrometer, a filter based spectrometer, a laser-based spectrometer, and an LED-based spectrometer.

5. A method as in claim 1, wherein the expected range of measurement variation includes variation due to one or more of: wavelength axis, line shape, resolution, intensity shifts, noise frequency content, and noise frequency bandwidth.

6. A method as in claim 1, wherein the expected range of measurement variation includes variation due to manufacturing tolerances in the optical interface with the sample.

7. A spectroscopic measurement device, having a calibration model produced according to the method of claim 1.
